(19) United States
(12) Patent Application Publication
(10) Pub. No.: US 2025/0195698 A1
(43) Pub. Date: Jun. 19, 2025

(54) GAS NANO/MICRO-BUBBLE COMPOSITIONS AND USES THEREOF

(71) Applicant: Lantheus Medical Imaging, Inc., North Billerica, MA (US)

(72) Inventors: Simon P. Robinson, Stow, MA (US); Bela Denes, Jacksonvill, OR (US)

(73) Assignee: Lantheus Medical Imaging, Inc., North Billerica, MA (US)

(21) Appl. No.: 18/836,615

(22) PCT Filed: Feb. 9, 2023
(86) PCT No.: PCT/US2023/062330

§ 371 Date: Aug. 7, 2024

Related U.S. Application Data
(60) Provisional application No. 63/308,487, filed on Feb. 9, 2022.

(51) Int. Cl. A61K 49/22; A61K 9/51

(52) U.S. Cl. CPC A61K 49/223; A61K 9/5192

(57) ABSTRACT

Provided herein are gas bubble populations optimized for certain imaging and therapeutic ultrasound uses, methods of preparing such populations, and methods of use thereof.

### RELATED APPLICATIONS

This application is a national stage filing under 35 U.S.C. 371 of International Patent Application Serial No. PCT/US2023/062330, filed Feb. 9, 2023, which claims the benefit of the filing date of U.S. Provisional Application No. 63/308,487 filed on Feb. 9, 2022, the entire contents of each of which are incorporated by reference herein.

### BACKGROUND OF INVENTION

Lipid-encapsulated gas bubbles can be formed by vigorous shaking of a container housing a lipid formulation such as a lipid suspension and a gas. The gas bubbles so formed have been used in imaging applications.

### SUMMARY OF INVENTION

This disclosure contemplates optimized populations of lipid-encapsulated gas bubbles, methods for preparing such populations, and methods of using such populations in combination with ultrasound in a variety of therapeutic applications as well as in imaging applications such as diagnostic imaging applications. These populations may comprise nanobubbles and/or microbubbles, and for the sake of brevity they are referred to herein as nano/microbubble populations. The efficacy of certain therapeutic and/or imaging (e.g., diagnostic imaging) ultrasound applications can be improved using the nano/microbubble populations provided by this disclosure. The disclosed preparation methods involve the use of optimized activation criteria and/or unique lipid formulations to form the selectively enriched nano/microbubble populations. Each optimized population may be prepared using unique activation parameters, including for example activation time and activation speed, and/or unique formulations.

The nano/microbubble populations provided herein may be used therapeutically together with ultrasound. These methods contemplate that the nano/microbubbles absorb ultrasound energy and then expand and compress (e.g., in the case of stable cavitation) or rupture (e.g., in the case of inertial cavitation). The degree to which the nano/microbubbles expand and compress, or rupture, is influenced in part by the ultrasound (wave) frequency and peak pressure differences together with the nano/microbubble size (typically measured in terms of diameter) and composition. Ultrasound frequencies and pressures that induce resonance in the nano/microbubbles with destruction (in the case of inertial cavitation) or without destruction (in the case of stable cavitation) can impart disruptive energy into the local environment that in turn can trigger a variety of intended consequences such as thrombus destruction and subsequent restoration of blood flow, blood-brain-barrier (BBB) disruption, tumor destruction, improved organ blood flow, etc. Further, in the case of inertial cavitation, the nano/microbubbles may be used to deliver a therapeutic agent to a region of interest including for example the brain or a solid cancer. Lipid-encapsulated gas microbubbles have been used extensively in contrast enhanced ultrasound (CEU) imaging. Some of the nano/microbubble populations provided herein, including those enriched for microbubbles having diameters of about 2 to about 6 microns or about 6 to about 10 microns, or about 2 to about 10 microns) also may be used together with ultrasound in imaging applications, such as those that traditional ultrasound contrast agents (UCA) have been used for in the past. These imaging applications include but are not limited to cardiovascular imaging (e.g., using a UCA in cases of suboptimal echocardiograms, in order to opacify the left ventricular chamber and to improve the delineation of the left ventricular endocardial border). The populations provided herein that are enriched for certain microbubbles (e.g., those having a diameter of about 2 microns to about 6 microns, or about 6 microns to about 10 microns) can provide better imaging quality than was previously possible, due to the increased proportion of these larger microbubbles.

This disclosure further provides populations of lipid-encapsulated gas nano/microbubble optimized for therapeutic use in combination with ultrasound, including low frequency ultrasound in the 2.25 MHz range and high frequency ultrasound in the 7.5 MHz range. These optimized populations have a different size and concentration profile than those made and used previously for imaging (e.g., diagnostic) and therapeutic applications.

Thus certain imaging and therapeutic methods provided herein involve use of the improved nano/microbubble populations in particular formulations, optionally administered at particular rates and/or doses, together with optimized ultrasound frequency, mechanical index (MI), pulse duration and/or number and/or timing of ultrasound applications.

Thus, in one aspect, this disclosure provides a composition comprising a population of lipid-encapsulated perfluorocarbon gas bubbles, wherein 75% or more of the lipid-encapsulated perfluorocarbon gas bubbles are nanobubbles having a diameter in the range of about 100 nanometers to less than or about 1 micron, and wherein the lipid-encapsulated perfluorocarbon gas bubbles comprise DPPC and PEG-DPPE. In some embodiments, about 80% or more of the lipid-encapsulated perfluorocarbon gas bubbles are nanobubbles having a diameter in the range of about 100 nanometers to less than or about 1 micron. The compositions may comprise 7.0×10⁹-2.5×10¹⁰ such nanobubbles per mL, or 1×10¹⁰-2.5×10¹⁰ such nanobubbles per mL.

In some embodiments of this and other aspects, the lipid-encapsulated perfluorocarbon gas bubbles comprise DPPA, DPPC and PEG-DPPE, optionally in a mole % ratio of about 10 to about 82 to about 8 including 10:82:8, or wherein the lipid formulation comprises DPPE, DPPC and PEG-DPPE, optionally in a mole % ratio of about 10 to about 82 to about 8 including 10:82:8. In some embodiments of this and other aspects, PEG-DPPE is PEG5000-DPPE. In some embodiments of this and other aspects, PEG-DPPE is MPEG5000-DPPE.

In some embodiments of this and other aspects, the lipid-encapsulated perfluorocarbon gas bubbles are lipid-encapsulated perfluoropropane gas bubbles. In these and other aspects and embodiments, other perfluorocarbon gases may be used as the perfluorcarbon gas including but not limited to perfluorpentane, perfluoroisopentane, perfluorobutane, perfluoropentene, and dodecafluoropentane. It is to be understood that the various aspects and embodiments described herein in relation to perfluorocarbon gas are equally applicable to other gases such as but not limited to sulfer (sulpher) hexafluoride gas.

In some embodiments of this and other aspects, the population of lipid-encapsulated perfluorocarbon gas bubbles is present in a non-aqueous lipid solution comprising DPPA, DPPC and PEG-DPPE, glycerol, propylene glycol, and less than or equal to 5% water (v/v), or wherein the population of lipid-encapsulated perfluorocarbon gas bubbles is present in a non-aqueous lipid solution comprising DPPE, DPPC and PEG-DPPE, glycerol, propylene glycol, and less than or equal to 5% water (v/v). In some embodiments of this and other aspects, the lipids are present in a combined concentration of about 3.75 mg/ml. In some embodiments of this and other aspects, the lipids are present in a combined concentration of about 2.5 mg/ml.

In some embodiments of this and other aspects, the population of lipid-encapsulated perfluorocarbon gas bubbles is present in an aqueous lipid suspension comprising glycerol and propylene glycol. In some embodiments of this and other aspects, the lipid suspension has a combined lipid concentration of about 0.75 mg/ml.

In some embodiments of this and other aspects, the population of lipid-encapsulated perfluorocarbon gas bubbles lacks DPPA.

In another aspect, this disclosure provides a method for forming lipid-encapsulated perfluorocarbon gas bubbles comprising activating a lipid formulation comprising DPPC and PEG-DPPE, glycerol and propylene glycol, in the presence of a perfluorocarbon gas, at a shaking rate and shaking time sufficient to yield a population of lipid-encapsulated perfluorocarbon gas bubbles wherein 75% or more of the gas bubbles are nanobubbles having a diameter in the range of about 100 nanometers to about 1 micron. About 80% or more of the lipid-encapsulated perfluorocarbon gas bubbles may be nanobubbles having a diameter in the range of about 100 nanometers to about 1 micron.

In some embodiments of this and other aspects, the lipid formulation comprises DPPA, DPPC and PEG-DPPE, optionally in a mole % ratio of about 10 to about 82 to about 8 including 10:82:8, or wherein the lipid formulation comprises DPPE, DPPC and PEG-DPPE, optionally in a mole % ratio of about 10 to about 82 to about 8 including 10:82:8. In some embodiments of this and other aspects, the perfluorocarbon gas is perfluoropropane gas.

In some embodiments, the lipid formulation is activated at a shaking rate of about 6000 rpm for a shaking time of about 50 seconds to about 90 seconds. In some embodiments, the lipid formulation is activated at a shaking rate of about 6000 rpm for a shaking time of about 50 seconds, about 60 seconds, about 75 seconds, or about 90 seconds.

In some embodiments of this and other aspects, the lipid formulation is present in a Wheaton 2 mL glass vial having an internal volume of about 3.8 ml, and comprising about 0.35 ml of lipid formulation, and about 3.45 ml of gas, or wherein the lipid formulation is present in a Wheaton 2 mL glass vial having an internal volume of about 3.8 ml, and comprising about 0.52 ml of lipid formulation, and about 3.28 ml of gas. In some embodiments of this and other aspects, the lipid formulation is present in a Wheaton 2 mL glass vial having an internal volume of about 3.8 ml, wherein about 9% of the internal volume is lipid occupied by lipid formulation prior to activation, or wherein about 14% of the internal volume is occupied by lipid formulation prior to activation. In these embodiments, the lipid formulation may be a non-aqueous lipid formulation.

In some embodiments of this and other aspects, the lipid formulation is an aqueous lipid suspension comprising DPPA, DPPC and PEG-DPPE, glycerol, and propylene glycol, or wherein the lipid formulation is an aqueous lipid suspension comprising DPPE, DPPC and PEG-DPPE, glycerol, and propylene glycol. In some embodiments of this and other aspects, the lipid formulation has a combined lipid concentration of about 0.75 mg/ml.

In some embodiments, the lipid formulation is activated in the presence of gas at a shaking rate of about 4530 rpm for a shaking time of about 90 seconds. In some embodiments, the lipid formulation is activated in the presence of gas at a shaking rate of about 6000 rpm for a shaking time of about 20 seconds to about 25 seconds.

In some embodiments, the lipid formulation lacks DPPA.

Also provided is a population of lipid-encapsulated perfluorocarbon gas bubbles formed according to any of the foregoing methods,

In another aspect, this disclosure provides a method of treating a subject having a condition, comprising administering to a subject having a condition an effective amount of a population of lipid-encapsulated perfluorocarbon gas bubbles, wherein 75% or more of the bubbles in the population are nanobubbles having a diameter in the range of about 100 nanometers to about 1 micron, and wherein the lipid-encapsulated perfluorocarbon gas bubbles comprise DPPC and PEG-DPPE, and applying ultrasound energy to a region of the subject affected by the condition.

In another aspect, this disclosure provides an improvement to a method of enhancing tissue perfusion in a subject comprising exposing the subject to therapeutic ultrasound, the improvement comprising administering to the subject a population of lipid-encapsulated perfluorocarbon gas bubbles wherein 75% or more of the bubbles in the population are nanobubbles having a diameter in the range of about 100 nanometers to about 1 micron before and/or during exposure to the therapeutic ultrasound.

In some embodiments of this and other aspects, the condition is presence of a thrombus/blood clot, vascular occlusion, retinal vein occlusion, a tumor, erectile dysfunction, Alzheimer's disease, Parkinson's disease. In some embodiments of this and other aspects, the condition is one that benefits from disruption of the blood-brain-barrier (BBB), optionally wherein a condition-specific therapeutic agent is also administered to the subject, before, during and/or after the population of gas bubbles and/or before, during and/or after exposure to the therapeutic ultrasound.

In some embodiments of this and other aspects, the lipid-encapsulated perfluorocarbon gas nanobubbles experience stable cavitation in vivo after application of the ultrasound energy. In some embodiments of this and other aspects, the lipid-encapsulated perfluorocarbon gas nanobubbles experience inertial cavitation in vivo after application of the ultrasound energy, optionally as detected and/or measured by emitted broadband radiofrequency.

Any of the afore-mentioned gas bubble populations may be used in the treatment of these conditions.

In another aspect, this disclosure provides a composition comprising a population of lipid-encapsulated perfluorocarbon gas bubbles, wherein 15% or more of microbubbles in the population have a diameter of about 2 microns to less than or about 6 microns, and wherein the lipid-encapsulated perfluorocarbon gas bubbles comprise DPPC and PEG-DPPE. In some embodiments, about 20% or more of the lipid-encapsulated perfluorocarbon gas microbubbles in the population have a diameter of about 2 microns to less than or about 6 microns. These compositions may comprise about 0.24×10⁹-2.44×10⁹ such bubbles per mL.

In some embodiments, the population of lipid-encapsulated perfluorocarbon gas bubbles is present in a non-aqueous lipid solution comprising DPPA, DPPC and PEG-DPPE, glycerol, propylene glycol, and less than or equal to 5% water (v/v), or wherein the population of lipid-encapsulated perfluorocarbon gas bubbles is present in a non-aqueous lipid solution comprising DPPE, DPPC and PEG-DPPE, glycerol, propylene glycol, and less than or equal to 5% water (v/v).

In some embodiments, the population of lipid-encapsulated perfluorocarbon gas bubbles is present in an aqueous lipid suspension comprising glycerol and propylene glycol.

In some embodiments, the population of lipid-encapsulated perfluorocarbon gas bubbles exhibits an acoustic attenuation, of 2.25 MHz ultrasound frequency, in the range of greater than about 1, optionally greater than about 1.2, optionally in the range of about 1 to about 1.7.

In another aspect, this disclosure provides a method for forming lipid-encapsulated perfluorocarbon gas bubbles comprising activating a lipid formulation comprising DPPC and PEG-DPPE, glycerol and propylene glycol, in the presence of a perfluorocarbon gas, at a shaking rate and a shaking time sufficient to yield a population of lipid-encapsulated perfluorocarbon gas microbubbles wherein 15% or more of the microbubbles have a diameter of about 2 microns to less than or about 6 microns.

In some embodiments, about 20% or more of the lipid-encapsulated perfluorocarbon gas microbubbles have a diameter of about 2 microns to less than or about 6 microns.

In some embodiments, the lipid formulation is a non-aqueous lipid solution comprising DPPA, DPPC and PEG-DPPE, glycerol and propylene glycol, and less than or equal to 5% water (v/v), or wherein the lipid formulation is a non-aqueous lipid solution comprising DPPE, DPPC and PEG-DPPE, glycerol and propylene glycol, and less than or equal to 5% water (v/v),

In some embodiments, the lipid formulation is activated at a shaking rate of about 6000 rpm for a shaking time of about 75 seconds to about 90 seconds. In some embodiments, the lipid formulation is activated at a shaking rate of about 6000 rpm for a shaking time of about 75 seconds or about 90 seconds.

In some embodiments, the lipid formulation is present in a Wheaton 2 mL glass vial having an internal volume of about 3.8 ml, and wherein the vial contains about 0.35 ml of lipid formulation and about 3.45 ml of gas, or wherein the vial contains about 0.52 ml of lipid formulation and about 3.28 ml of gas. In some embodiments, the lipid formulation is present in a Wheaton 2 mL glass vial having an internal volume of about 3.8 ml, wherein about 9% of the internal volume of the vial is occupied by lipid formulation prior to activation, or wherein about 14% of the internal volume of the vial is occupied by lipid formulation prior to activation.

In some embodiments, prior to activating, a solution of propylene glycol and glycerol, in equal volume proportions, is combined with the lipid formulation.

In some embodiments, the lipid formulation is activated at a shaking rate of about 6000 rpm for a shaking time of about 60 seconds.

In some embodiments, the lipid formulation is an aqueous lipid suspension comprising DPPA, DPPC, PEG-DPPE, glycerol and propylene glycol, or wherein the lipid formulation is an aqueous lipid suspension comprising DPPE, DPPC, PEG-DPPE, glycerol and propylene glycol.

In some embodiments, the lipid formulation is activated at a shaking rate of about 6000 rpm for a shaking time of about 30 seconds.

A population of lipid-encapsulated perfluorocarbon gas bubbles formed according to any of the foregoing methods is also provided.

In another aspect, this disclosure provides a method of treating a subject having a condition, comprising administering to a subject having a condition an effective amount of a population of lipid-encapsulated perfluorocarbon gas bubbles, wherein 15% or more of microbubbles in the population have a diameter of about 2 microns to less than or about 6 microns, and wherein the lipid-encapsulated perfluorocarbon gas bubbles comprise DPPC and PEG-DPPE, and applying ultrasound energy, optionally at a frequency of about 1 to about 10 MHz, to a region of the subject affected by the condition.

In another aspect, this disclosure provides a method of improving therapeutic or diagnostic ultrasound, the improvement comprising administering to the subject a population of lipid-encapsulated perfluorocarbon gas bubbles wherein 15% or more of the microbubbles in the population have a diameter in the range of about 2 microns to less than or about 6 microns, before or during exposure to ultrasound, optionally at a frequency of about 1 to about 10 MHz. In some embodiments, about 20% or more of the lipid-encapsulated perfluorocarbon gas microbubbles have a diameter of about 2 microns to less than or about 6 microns.

In some embodiments of this and other aspects, the condition is cardiac atherosclerosis, myocardial infarct, vascular occlusion, stroke, peripheral artery disease.

In some embodiments of this and other aspects, the lipid-encapsulated perfluorocarbon gas bubbles experience stable cavitation in vivo after application of the ultrasound energy, optionally as detected and/or measured with harmonic frequency emission. In some embodiments of this and other aspects, the lipid-encapsulated perfluorocarbon gas bubbles experience inertial cavitation in vivo after application of the ultrasound energy, optionally as detected and/or measured with broadband emission.

In some embodiments of this and other aspects, the population of lipid-encapsulated perfluorocarbon gas bubbles has an acoustic attenuation, at 2.25 MHz ultrasound frequency, of greater than about 1.0. In some embodiments of this and other aspects, the population of lipid-encapsulated perfluorocarbon gas microspheres has an acoustic attenuation, at 2.25 MHZ ultrasound frequency, of about 1.0 to about 1.5, or about 1.0 to about 2.0.

In another aspect, this disclosure provides a composition comprising a population of lipid-encapsulated perfluorocarbon gas bubbles, wherein 4% or more of microbubbles in the population have a diameter of about 6 microns to about 10 microns, and wherein the lipid-encapsulated perfluorocarbon gas bubbles comprise DPPC and PEG-DPPE. In some embodiments, about 10% or more of the lipid-encapsulated perfluorocarbon gas microbubbles in the population have a diameter of about 6 microns to about 10 microns. These compositions may comprise about 0.1×10⁸-0.32×10⁸ such bubbles per mL.

In some embodiments, the population of lipid-encapsulated perfluorocarbon gas bubbles exhibits an acoustic attenuation of 2.25 MHz ultrasound frequency in the range of greater than about 1, optionally greater than about 1.2, optionally in the range of about 1 to about 1.7. In some embodiments, the population of lipid-encapsulated perfluorocarbon gas bubbles lacks DPPA.

In another aspect, this disclosure provides a method for forming lipid-encapsulated perfluorocarbon gas bubbles comprising activating a lipid formulation comprising DPPC and PEG-DPPE, glycerol and propylene glycol, in the presence of a perfluorocarbon gas, at a shaking rate and a shaking time sufficient to yield a population of lipid-encapsulated perfluorocarbon gas microbubbles wherein 4% or more of the microbubbles have a diameter of about 6 microns to about 10 microns. In some embodiments, about 10% or more of the lipid-encapsulated perfluorocarbon gas microbubbles have a diameter of about 6 microns to about 10 microns.

In some embodiments, the lipid formulation is a non-aqueous lipid solution comprising DPPA, DPPC and PEG-DPPE, glycerol and propylene glycol, and less than or equal to 5% water (v/v), or wherein the lipid formulation is a non-aqueous lipid solution comprising DPPA, DPPC and PEG-DPPE, glycerol and propylene glycol, and less than or equal to 5% water (v/v). In some embodiments, the lipid formulation is present in a Wheaton 2 mL glass vial having an internal volume of about 3.8 ml, and wherein the vial contains about 0.35 ml of lipid formulation and about 3.45 ml of gas, or wherein the vial contains about 0.52 ml of lipid formulation and about 3.28 ml of gas.

In some embodiments, the lipid formulation is present in a Wheaton 2 mL glass vial having an internal volume of about 3.8 ml, wherein about 9% of the internal volume of the vial is occupied by lipid formulation prior to activation, or wherein about 14% of the internal volume of the vial is occupied by lipid formulation prior to activation.

In some embodiments, the lipid formulation is activated at a shaking rate of about 6000 rpm for a shaking time of about 60 seconds.

In some embodiments, the lipid formulation is an aqueous lipid suspension comprising DPPA, DPPC, PEG-DPPE, glycerol and propylene glycol, or wherein the lipid formulation is an aqueous lipid suspension comprising DPPE, DPPC, PEG-DPPE, glycerol and propylene glycol.

In another aspect, this disclosure provides a method of treating a subject having a condition, comprising administering to a subject having a condition an effective amount of a population of lipid-encapsulated perfluorocarbon gas bubbles, wherein 4% or more of microbubbles in the population have a diameter of about 6 microns to about 10 microns, and wherein the lipid-encapsulated perfluorocarbon gas bubbles comprise DPPC and PEG-DPPE, and applying ultrasound energy at a frequency of about 1 to about 10 MHz to a region of the subject affected by the condition.

In another aspect, this disclosure provides a method of improving therapeutic or diagnostic ultrasound, the improvement comprising administering to the subject a population of lipid-encapsulated perfluorocarbon gas bubbles wherein 4% or more of the microbubbles in the population have a diameter in the range of about 6 microns to about 10 microns, before or during exposure to ultrasound at a frequency of about 1 to about 10 MHz. In some embodiments, wherein 10% or more of the lipid-encapsulated perfluorocarbon gas microbubbles have a diameter of about 6 microns to about 10 microns.

In some embodiments of this and other aspects, the condition is cardiac atherosclerosis, myocardial infarct, vascular occlusion, stroke, peripheral artery disease.

In some embodiments, the population of lipid-encapsulated perfluorocarbon gas bubbles has an acoustic attenuation of 2.25 MHz ultrasound frequency of greater than about 1.0.

In some embodiments, the population of lipid-encapsulated perfluorocarbon gas microspheres has an acoustic attenuation of 2.25 MHz ultrasound frequency of about 1.0 to about 1.5, or about 1.0 to about 2.0.

In another aspect, this disclosure provides a method of ultrasound imaging a subject, comprising administering to a subject any of the foregoing populations of lipid-encapsulated perfluorocarbon gas bubbles or any of the lipid-encapsulated perfluorocarbon gas bubbles made by any of the foregoing methods, applying ultrasound energy at a frequency in the range of about 1 to about 10 MHz, and obtaining an ultrasound image of the subject or a region of the subject.

In some embodiments, the perfluorocarbon gas is perfluoropropane gas.

In some embodiments, the ultrasound energy is at a frequency of about 1.25 MHz or about 2.25 MHz.

In another aspect, this disclosure provides a method for forming lipid-encapsulated perfluorocarbon gas bubbles comprising activating, in the presence of a perfluorocarbon gas, a non-aqueous lipid formulation comprising less than or equal to 5% water (v/v), DPPC, PEG-DPPE, glycerol, propylene glycol, and either DPPA or DPPE, at a shaking rate of about 6000 rpm for about 30 to about 90 seconds, optionally for about 50, about 60, about 75, or about 90 seconds, thereby forming a population of lipid-encapsulated perfluoropropane gas microspheres.

In some embodiments, the lipid formulation and gas are present in a vial, optionally a 2 mL vial, and the lipid formulation occupies about 9% or about 14% of the internal volume of the vial.

In another aspect, this disclosure provides a method for forming lipid-encapsulated perfluorocarbon gas bubbles comprising activating, in the presence of a perfluorocarbon gas, a non-aqueous lipid formulation comprising less than or equal to 5% water (v/v), DPPC, PEG-DPPE, glycerol, propylene glycol, and either DPPA or DPPE, at a shaking rate of about 4950 rpm for about 125 seconds, thereby forming a population of lipid-encapsulated perfluoropropane gas microspheres. In some embodiments, the lipid formulation and gas are present in a vial, optionally a 2 mL vial, and the lipid formulation occupies about 9% or about 14% of the internal volume of the vial.

In another aspect, this disclosure provides a method for forming lipid-encapsulated perfluorocarbon gas bubbles comprising activating, in the presence of a perfluorocarbon gas, an aqueous lipid formulation comprising DPPC and PEG-DPPE, and glycerol and propylene glycol, and either DPPA or DPPE, at a shaking rate of about 6000 rpm for about 5 to about 90 seconds, optionally for about 5, about 10, about 20, about 25, about 30, about 40, about 45, about 50, about 55, about 60, about 70, about 80, or about 90 seconds, thereby forming a population of lipid-encapsulated perfluoropropane gas microspheres.

In another aspect, this disclosure provides a method for forming lipid-encapsulated perfluorocarbon gas bubbles comprising activating, in the presence of a perfluorocarbon gas, an aqueous lipid formulation comprising DPPC and PEG-DPPE, glycerol and propylene glycol, and either DPPA or DPPE, at a shaking rate of about 4530 rpm for about 90 seconds, thereby forming a population of lipid-encapsulated perfluoropropane gas microspheres.

In another aspect, this disclosure provides a method for forming lipid-encapsulated perfluorocarbon gas bubbles comprising activating, in the presence of a perfluorocarbon gas, an aqueous lipid formulation comprising DPPC and PEG-DPPE, glycerol and propylene glycol, and either DPPA or DPPE, at a shaking rate of about 4530 rpm for about 10 to about 20 seconds, thereby forming a population of lipid-encapsulated perfluoropropane gas microspheres.

In another aspect, this disclosure provides a method for forming lipid-encapsulated perfluororpropane nanodroplets comprising condensing a population of lipid-encapsulated perfluoropropane gas microspheres by applying sufficient pressure to condense the microbubbles into nanodroplets, wherein the population of gas microspheres are condensed at room temperature.

In some embodiments, applying pressure comprises applying manual pressure. In some embodiments, applying pressure comprises applying pressure in the range of about 6 to about 12 atm.

In some embodiments, the population of lipid-encapsulated perfluoropropane gas microspheres is provided in a syringe, optionally a 3 mL syringe or a 5 mL syringe, and applying pressure comprises applying manual pressure through a plunger. In some embodiments, the population of lipid-encapsulated perfluoropropane gas microspheres is prepared by activating a lipid formulation comprising DPPA, DPPC and PEG5000-DPPE in the presence of perfluoropropane. In some embodiments, the population of lipid-encapsulated perfluoropropane gas microspheres is prepared by activating a lipid formulation comprising DPPC, DPPE and PEG5000-DPPE in the presence of perfluoropropane. In some embodiments, the lipid formulation is a non-aqueous lipid formulation and optionally wherein activating a lipid formulation comprises activating at about 4950 rpm for 45 seconds, or about 6000 rpm to about 6200 rpm for about 60 seconds, or about 6600 rpm for about 15 seconds to about 30 seconds. In some embodiments, the lipid formulation is an aqueous lipid formulation and optionally wherein activating a lipid formulation comprises activating at about 4530 rpm for 45 seconds.

In another aspect, this disclosure provides a method of diagnosis or therapy in a subject comprising administering to a subject a population of lipid-encapsulated nanodroplets and exposing the subject to ultrasound energy, optionally at a targeted location, wherein the population of lipid-encapsulated nanodroplets is prepared using the method described herein.

In some embodiments, the nanodroplets comprise a lipid shell made of DPPA, DPPC and PEG5000-DPPE and a liquid center of perfluoropane. In some embodiments, the nanodroplets comprise a lipid shell made of DPPC, DPPE and PEG5000-DPPE and a liquid center of perfluoropane. In some embodiments, the nanodroplets lack DPPA. In some embodiments, the nanodroplets are about 100 to about 200 nanometers in size.

In another aspect, this disclosure provides a method of treating a subject having erectile dysfunction comprising administering to a subject in need thereof an effective amount of lipid-encapsulated gas bubbles or nanodroplets, and performing ultrasound on the genital area of the subject.

In some embodiments, the subject having erectile dysfunction has mild-to-moderate erectile dysfunction as measured by an HEF-5 or an IIEF-15 score. In some embodiments, treatment is evidenced by an increase in an IIEF-5 or an IIEF-15 score following infusion and ultrasound, optionally within 1-7 days following infusion and ultrasound.

In some embodiments, the subject is administered lipid-encapsulated gas bubbles that comprise a lipid shell comprising DPPA, DPPC and MPEG5000-DPPE and a perfluorpropane gas center, optionally wherein the lipid-encapsulated gas bubbles are present in an activated DEFINITY formulation or an activated DEFINITY RT formulation. In some embodiments, the subject is administered lipid-encapsulated gas bubbles that comprise a lipid shell comprising DPPC, DPPE and MPEG5000-DPPE and a perfluorpropane gas center. In some embodiments, the subject is administered lipid-encapsulated nanodroplets that comprise a lipid shell comprising DPPA, DPPC and MPEG5000-DPPE and a center comprising perfluorpropane in liquid form, optionally wherein the lipid-encapsulated nanodroplets are formed by condensation of lipid-encapsulated gas bubbles present in an activated DEFINITY formulation or an activated DEFINITY RT formulation. In some embodiments, the subject is administered lipid-encapsulated nanodroplets that comprise a lipid shell comprising DPPC, DPPE and MPEG5000-DPPE and a center comprising perfluorpropane in liquid form.

In some embodiments, ultrasound comprises high intensity ultrasound, optionally at a frequency in the range of 1 to 2 MHZ, and further optionally at a mechanical index of about 1 to about 1.5 or about 1 to about 1.3.

In some embodiments, the method is performed repeatedly at regularly spaced intervals, optionally wherein the method is performed once or twice a week for one or more weeks.

These and other aspects and embodiments of this disclosure will be discussed in greater detail herein.

### BRIEF DESCRIPTION OF DRAWINGS

FIG. 1 provides photographs of a Norm Jet syringe containing DEFINITY RT activated at 6000 rpm for 60 seconds prior to the application of hand pressure (top), following the application of hand pressure and prior to the release of such pressure (middle), and following the release of pressure through removal of the cap (bottom). The FIGURE demonstrates the milky white appearance of activated DEFINITY RT prior to applying hand pressure and the colorless appearance of the activated DEFINITY RT following applying hand pressure. The change in appearance correlates with the selective reduction in number of bubbles that are >500 nm, and remains even after the cap is removed and the pressure is released from the syringe.

### DETAILED DESCRIPTION OF INVENTION

This disclosure provides nano/microbubble compositions having improved therapeutic and imaging properties, methods of preparing such compositions including specific devices to be used in such preparatory methods, and methods of use in therapeutic and imaging applications. The preparatory methods result in an optimized nano/microbubble composition to be used together with ultrasound in a variety of therapeutic applications and/or a variety of imaging applications. Different therapeutic applications may require different optimized nano/microbubble compositions. It has been found unexpectedly that such optimized nano/microbubble compositions may be generated by particular manipulation of lipid formulations such as lipid solutions and lipid suspensions and/or particular activation parameters. As an example, certain of the optimized nano/microbubble compositions provided herein may be prepared using unique lipid formulation activation parameters, including activation time, and activation speed (or rate), as well as unique manipulation of lipid formulations.

### Nano/Microbubble Populations

The nano/microbubble populations provided herein are defined by their size and concentration profiles, their acoustic attenuation properties, and/or their preparatory methods. Their utility in therapeutic ultrasound applications depend in part on their size and/or acoustic attenuation profiles.

As used herein, the size profile of a nano/microbubble population means the proportion of bubbles in the population that have a particular size. The size profile may include the proportions of bubbles of different sizes, in some instances. The following describes certain nano/microbubble populations that are defined by their size profile.

One aspect of this disclosure provides a population of lipid-encapsulated gas bubbles, wherein about 75% or more of the lipid-encapsulated gas bubbles are nanobubbles having a diameter in the range of about 100 nanometers to less than or about 1 micron (i.e., a diameter of about 100 nanometers to less than or about 1 micron, as intended herein). In some instances, the diameter is in the range of about 200 nanometers to less than or about 1 micron, or about 300 nanometers to less than or about 1 micron, or about 500 nanometers to less than or about 1 micron. In related instances, the upper end of the diameter range is less than 1 micron, or about 950 nanometers, or about 900 nanometers, or about 850 nanometers, or about 800 nanometers, or about 750 nanometers, or about 700 nanometers. Any combination of these lower and upper ends is contemplated, including but not limited to about 200 nanometer to about 800 nanometers or about 500 nanometers to less than 1 micron. Thus, as an example, 75% or more of the lipid-encapsulated gas bubbles may be nanobubbles having a diameter in the range of about 100 nanometers to less than or about 1 micron, or in the range of about 200 nanometers to about 800 nanometers. All other lower and upper ends combinations are contemplated by this disclosure.

Such a population may be referred to as being enriched for lipid-encapsulated gas bubbles having diameters in any of the afore-mentioned ranges. Thus, a population of lipid-encapsulated gas bubbles, wherein about 75% or more of the lipid-encapsulated gas bubbles have a diameter in the range of about 100 nanometers to less than or about 1 micron, is considered enriched for lipid-encapsulated gas bubbles having a diameter in the range of about 100 nanometers to less than or about 1 micron. About 75%, about 80%, about 85%, about 90%, about 95% or more of the lipid-encapsulated gas bubbles in these enriched populations may have a diameter in the range of about 100 nanometers to less than or about 1 micron, or in any other of the specified ranges including for example a range of about 200 nanometers to less than or about 1 micron, or a range of about 300 nanometers to less than or about 1 micron, or a range of about 400 nanometers to less than or about 1 micron, or a range of about 500 nanometers to less than or about 1 micron, including ranges having the afore-mentioned lower ends and upper ends of less than 1 micron, or about 950 nanometers, or about 900 nanometers, or about 850 nanometers, or about 800 nanometers, or about 750 nanometers, or about 700 nanometers.

As discussed below, nanobubble-enriched populations (relative to prior art populations) can be used in new therapeutic applications or can improve the therapeutic efficacy of existing therapeutic applications.

Another aspect of this disclosure provides a population of lipid-encapsulated gas bubbles, wherein about 15% or more of the lipid-encapsulated gas microbubbles in the population have a diameter in the range of about 2 microns to less than or about 6 microns (i.e., a diameter of about 1 micron to less than or about 6 microns, as intended herein). In some instances, the diameter is in the range of about 2.5 microns to less than or about 6 microns, or about 3 microns to less than or about 6 microns, or about 3.5 microns to less than or about 6 microns. In related instances, the upper end of the diameter range is less than 6 microns, or about 5.5 microns, or about 5 microns, or about 4.5 microns, or about 4 microns. Any combination of these lower and upper ends is contemplated. Thus, as an example, 15% or more of the lipid-encapsulated gas microbubbles in the population may have a diameter in the range of about 2 microns to less than 6 microns, or in the range of about 2 microns to about 5.5 microns. All other lower and upper ends combinations are contemplated by this disclosure.

Such a population may be referred to as being enriched for lipid-encapsulated gas microbubbles having diameters in any of the afore-mentioned ranges. Thus, a population of lipid-encapsulated gas bubbles, wherein 15% or more of the lipid-encapsulated gas microbubbles in the population have a diameter in the range of about 2 microns to less than or about 6 microns, is considered enriched for lipid-encapsulated gas bubbles having a diameter in the range of about 2 microns to less than or about 6 microns. About 15%, about 20%, about 25%, about 30%, about 35% or more of the lipid-encapsulated gas microbubbles in these enriched populations may have a diameter in the range of about 2 microns to less than or about 6 microns, or in any other of the specified ranges including for example a range of about 2.5 microns to less than or about 6 microns, or a range of about 3 microns to less than or about 6 microns, or a range of about 3.5 microns to less than or about 6 microns, including ranges having the afore-mentioned lower ends and upper ends of less than 6 microns, or about 5.5 microns, or about 5 microns, or about 4.5 microns, or about 4 microns.

As discussed below, microbubble populations enriched in microbubbles in the about 2 to about 6 micron diameter range (relative to prior art populations) can be used in new therapeutic or imaging applications or can improve the efficacy of existing therapeutic or imaging applications. Another aspect of this disclosure provides a population of lipid-encapsulated gas bubbles, wherein about 4% or more of the lipid-encapsulated gas microbubbles in the population have a diameter in the range of about 6 microns to about 10 microns (i.e., a diameter of about 6 microns to about 10 microns, as intended herein). In some instances, the diameter is in the range of about 6.5 microns to about 10 microns, or about 7 microns to about 10 microns, or about 8 microns to about 10 microns. In related instances, the upper end of the diameter range is about 9.5 microns, or about 9.0 microns, or about 8.5 microns, or about 8.0 microns, or about 7.5 microns. Any combination of these lower and upper ends is contemplated. Thus, as an example, about 4% or more of the lipid-encapsulated gas microbubbles in the population may have a diameter in the range of about 6 microns to about 10 microns, or in the range of about 6 microns to about 9.5 microns. All other lower and upper ends combinations are contemplated by this disclosure.

Such a population may be referred to as being enriched for lipid-encapsulated gas microbubbles having diameters in any of the afore-mentioned ranges. Thus, a population of lipid-encapsulated gas bubbles, wherein 4% or more of the lipid-encapsulated gas microbubbles in the population have a diameter in the range of about 6 microns to about 10 microns, is considered enriched for lipid-encapsulated gas bubbles having a diameter in the range of about 6 microns to about 10 microns. About 4%, about 5%, about 7%, about 10%, about 12% or more of the lipid-encapsulated gas microbubbles in these enriched populations may have a diameter in the range of about 6 microns to about 10 microns, or in any other of the specified ranges including for example a range of about 6.5 microns to about 10 microns, or a range of about 7 microns to about 10 microns, or a range of about 7.5 microns to about 10 microns, including ranges having the afore-mentioned lower ends and upper ends of about 9.5 microns, or about 9 microns, or about 8.5 microns, or about 8 microns, or about 7.5 microns.

As discussed below, microbubble populations enriched in microbubbles in the about 6 to about 10 micron diameter range (relative to prior art populations) can be used in new therapeutic or imaging applications or can improve the efficacy of existing therapeutic or imaging applications,

### Lipid Formulations, Aqueous and Non-Aqueous

The lipid formulations used to form the gas nano/microbubbles of this disclosure may be non-aqueous lipid formulations, defined as having less than or equal to 5% water (w/w). These non-aqueous lipid formulations may sometimes be referred to herein as lipid solutions. Alternatively, the lipid formulations may be aqueous lipid formulations, defined as having greater than 5% water (w/w). These aqueous lipid formulations may sometimes be referred to herein as lipid suspensions. Both types of lipid formulations may be activated as described herein. The nano/microbubble enriched populations may be formed using aqueous lipid formulations or non-aqueous lipid formulations, together with a gas.

Suitable lipid formulations include one, two, three or more lipids. Suitable lipids are provided below. One of more such lipids may be conjugated to polyethylene glycol (PEG). One example of an aqueous lipid formulation comprises lipids DPPA, DPPC and MPEG5000-DPPE in a 10:82:8 mole % ratio, and a combined lipid concentration of about 0.75 mg/ml, as well as propylene glycol and glycerol in an aqueous solution. Activation of this aqueous lipid formulation in the presence of perfluoropropane gas (also known as perflutren or octafluoropropane) at 4530 rpm for 45 seconds yields about 10¹⁰ nanobubbles and about 5×10⁹ microbubbles. About 66% of the bubbles produced following activation of this aqueous lipid formulation are nanobubbles. About 4% of the microbubbles formed have a diameter in the range of about 2 to about 6 microns.

Other aqueous lipid formulations are similar to the afore-mentioned formulation but differ in their lipid profiles. For example, in some aqueous lipid formulations, DPPA, DPPC and DPPE may be used in molar percentages of about 77 to about 90 mole % DPPC, about 5 to about 15 mole % DPPA, and about 5 to about 15 mole % DPPE, including DPPE-MPEG5000 such as MPEG5000-DPPE. Similar ratios may be used in non-aqueous lipid formulations as well.

Another example of an aqueous lipid formulation comprises phosphatidylcholine (PC), phosphatidylethanolamine (PE), and dipalmitoylphosphatidylethanolamine (DPPE) and specifically lacks DPPA. The PE may be PE-PEG and/or the DPPE may be DPPE-PEG. For example, the lipid formulation may comprise lipids DPPC, DPPE and DPPE-PEG. These may be present in a mole percent ratio of 82:10:8 (DPPC:DPPE:DPPE-PEG). These lipids may be used in other molar percentages including about 77 to about 90 mole % DPPC, about 5 to about 15 mole % DPPE, and about 5 to about 15 mole % DPPE-PEG. The combined lipid concentration may range from about 0.75 mg/ml to about 1.5 mg/ml. These lipids may be suspended in propylene glycol, glycerol and saline, or any combination thereof. These formulations may be activated in the presence of a perfluorocarbon gas such as perflutren, or any other suitable gas.

One example of a non-aqueous lipid formulation comprises DPPA, DPPC and MPEG5000-DPPE in a 10:82:8 mole % ratio, and a combined lipid concentration of about 3.75 mg/ml, in propylene glycol and glycerol and less than or equal to 5% water (w/w). Activation of this non-aqueous lipid formulation in the presence of perfluoropropane gas at 4950 rpm for 45 seconds yields about 3×10⁹ nanobubbles and about 2.5-3×10⁹ microbubbles. About 50% of the bubbles produced following activation of this lipid formulation are nanobubbles. About 12% of the microbubbles formed have a diameter in the range of about 2 to less than or about 6 microns, and about 1.2% of the microbubbles formed have a diameter in the range of about 6 to about 10 microns.

Other non-aqueous lipid formulations are described in published PCT Application PCT/US2015/067615, the entire contents of which are incorporated by reference herein.

Another example of a non-aqueous lipid formulation comprises phosphatidylcholine (PC), phosphatidylethanolamine (PE), and dipalmitoylphosphatidylethanolamine (DPPE), and optionally specifically lacks DPPA. The PE may be PE-PEG and/or the DPPE may be DPPE-PEG. For example, the lipid formulation may comprise lipids DPPC, DPPE and DPPE-PEG. These may be present in a mole percent ratio of 82:10:8 (DPPC:DPPE:DPPE-PEG). These lipids may be used in other molar percentages including about 77 to about 90 mole % DPPC, about 5 to about 15 mole % DPPE, and about 5 to about 15 mole % DPPE-PEG. The combined lipid concentration may range from about 0.75 mg/ml to about 1.5 mg/ml. These lipids may be suspended in propylene glycol, glycerol, or propylene glycol and glycerol. These formulations may be activated in the presence of a perfluorocarbon gas such as perflutren, or any other suitable gas.

The non-aqueous lipid formulations may comprise lipids in propylene glycol, or glycerol, or propylene glycol and glycerol, provided the water content is less than or about 5% by weight (i.e., weight of water to the weight of the combination of lipids and propylene glycol and/or glycerol). In some instances, the non-aqueous lipid formulation comprises less than 5% water (w/w), 1-4% water (w/w), 1-3% water (w/w), 2-3% water (w/w), or 1-2% water (w/w). In some instances, the non-aqueous lipid formulation comprises less than 1% water (w/w). The water content may be measured at the end of manufacture (and prior to long term storage) or it may be measured after storage, including long term storage, and just before use.

The lipid formulations also may be salt-free (i.e., they do not contain any salts other than lipid counter-ions). More specifically, and as an example, lipids such as DPPA and DPPE are typically provided as sodium salts. As used herein, a salt-free lipid formulation may comprise such counter-ions (e.g., sodium if DPPA and/or DPPE are used) but they do not contain other ions. In some instances, the non-aqueous lipid formulation is free of sodium chloride or chloride.

The lipid formulation may comprise a buffer. The buffer may be an acetate buffer, a benzoate buffer, a salicylate buffer, a diethanolamine buffer, a triethanolamine buffer, a borate buffer, a carbonate buffer, a glutamate buffer, a succinate buffer, a malate buffer, a tartrate buffer, a glutarate buffer, an aconite buffer, a citrate buffer, a lactate buffer, a glycerate buffer, a gluconate buffer, or a tris buffer, although it is not so limited. Non-phosphate buffers may be preferred in some instances due to their dissolution profiles in the non-aqueous solvents such as propylene glycol and/or glycerol. In some instances, a phosphate buffer may be used (e.g., following or concurrent with addition of saline or other aqueous diluent).

In some embodiments, the non-aqueous lipid formulation comprises, consists of, or consists essentially of (a) one or more lipids, (b) propylene glycol, or glycerol, or propylene glycol and glycerol, and (c) a non-phosphate buffer.

In some instances, the lipid concentration in the lipid formulations may range from about 0.1 mg to about 20 mg per mL, including about 0.6 mg to about 10 mg per mL and about 0.6 mg to about 7.5 mg per mL. In some embodiments, the lipid concentration may range from about 0.75 mg to about 7.5 mg lipid per mL, including about 0.75 mg to about 3.75 mg lipid per mL, or about 0.75 mg to about 2.5 mg lipid per mL, or about 2.5 mg to about 7.5 mg lipid per mL, or about 3.75 mg to about 7.5 mg lipid per mL, or about 2.5 mg to about 3.75 mg lipid per mL. In some instances, the lipid concentration is about 2 mg to about 4 mg per mL, or about 2.5 mg to about 3.75 mg per mL, or about 3.75 mg to about 7.5 mg of total lipid per mL.

As an example, the lipid concentration in non-aqueous lipid formulations may range from about 0.1 mg to about 10 mg lipid per mL of propylene glycol and/or glycerol (combined), including about 1 mg to about 5 mg lipid per mL of propylene glycol and/or glycerol (combined), or about 2 mg to about 4 mg lipid per mL of propylene glycol and/or glycerol (combined), or about 2.5 mg to about 3.75 mg lipid per mL of propylene glycol and/or glycerol (combined).

As another example, the lipid concentration in aqueous lipid formulations may range from about 0.1 mg to about 10 mg lipid per mL, including about 0.1 mg to about 5 mg lipid per mL, or about 0.3 mg to about 1 mg lipid per mL, or about 0.5 mg to about 1 mg lipid per mL, or about 0.75 mg lipid per ml.

Prior to administration, the nano/microbubbles may be diluted with an aqueous diluent, and such aqueous diluent may comprise salts such as but not limited to sodium chloride, and thus may be regarded as saline or a saline solution.

The aqueous diluent may comprise a buffer such as a phosphate buffer, and thus may be regarded as a buffered aqueous diluent. The aqueous diluent may be a buffered saline solution.

The non-aqueous lipid formulation may comprise a buffer such as a non-phosphate buffer, examples of which are provided herein. The lipid formulation and the aqueous diluent may both comprise a buffer. In typical embodiments, either the lipid formulation or the aqueous diluent comprises a buffer, but not both. The buffer concentration will vary depending on the type of buffer used, as will be understood and within the skill of the ordinary artisan to determine. The buffer concentration in the lipid formulation may range from about 1 mM to about 100 mM. In some instances, the buffer concentration may be about 1 mM to about 50 mM, or about 1 mM to about 20 mM, or about 1 mM to about 10 mM, or about 1 mM to about 5 mM, including about 5 mM.

In some embodiments, the aqueous diluent comprises glycerol, a buffer such as phosphate buffer, salt(s) and water. Such an aqueous diluent may be used with a non-aqueous lipid formulation that lacks glycerol. In some embodiments, the lipid formulation further comprises saline (salt(s) and water combined) and glycerol in a weight ratio of 8:1. In some embodiments, the aqueous diluent comprises propylene glycol, a buffer such as phosphate buffer, salt(s) and water. Such an aqueous diluent may be used with a non-aqueous lipid formulation that lacks propylene glycol.

In some embodiments, the aqueous diluent comprises a buffer such as phosphate buffer, salt(s) and water. Such an aqueous diluent may be used with a non-aqueous lipid formulation that comprises both propylene glycol and glycerol.

The nano/microbubble population to be administered, typically intravenously, to a subject including a human subject may have a pH in a range of 4-8 or in a range of 4.5-7.5. In some instances, the pH may be in a range of about 6 to about 7.5, or in a range of 6.2 to about 6.8. In still other instances, the pH may be about 6.5 (e.g., 6.5+/−0.5 or +/−0.3). In some instances, the pH may be in a range of 5 to 6.5 or in a range of 5.2 to 6.3 or in a range of 5.5 to 6.1 or in a range of 5.6 to 6 or in a range of 5.65 to 5.95. In still another instance, the pH may be in a range of about 5.7 to about 5.9 (e.g., +/−0.1 or +/−0.2 or +/−0.3 cither or both ends of the range). In another instance, the pH may be about 5.8 (e.g., 5.8+/−0.15 or 5.8+/−0.1).

### Methods of Forming Nano/Microbubble Enriched Populations, Activation

The lipid-encapsulated gas nano/microbubbles of this disclosure are prepared by activating a lipid formulation such as a lipid solution or a lipid suspension. Activation is a process whereby the lipid formulation, housed in a container such as a vial, is physically agitated (e.g., vigorously shaken) in the presence of a gas of interest, to form lipid-encapsulated gas nano/microbubbles.

This disclosure provides methods for forming suitable nano/microbubble populations by uniquely activating lipid formulations at sufficient rates and for sufficient times. It has been found that the optimized nano/microbubble populations provided herein are obtained from lipid formulations using particular activation parameters. Some of these specific activation parameters are markedly different from the activation parameters of existing lipid formulations used to form microbubbles for diagnostic ultrasound applications.

As one example, it has been found that non-aqueous lipid formulations provided herein must be activated at higher speeds (or rates) for longer time periods time to generate certain enriched nano/microbubble populations of this disclosure. For example, certain of these non-aqueous lipid formulations generate microbubbles for diagnostic ultrasound applications when activated at 4950 rpm for 45 seconds. However, in order to generate certain of the nano/microbubble populations provided herein, these formulations, examples of which are provided in Example 2, must be activated at significantly higher rates, including at about 6000 rpm, for longer times, ranging from about 50 to about 90 seconds, including about 75 to about 90 seconds.

Certain enriched nano/microbubble populations therefore may be formed by activating non-aqueous formulations, such as described in Example 2, for a shaking time of about 50 second to about 90 seconds at a shaking rate of 6000 rpm+/−10%, or +/−5%, or +/−1%, and/or at a shaking rate in the range of about 5000 rpm to about 7000 rpm, or about 5500 rpm to about 6500 rpm, or about 5800 rpm to about 6200 rpm, or about 5900 to about 6100 rpm, or about 5950 rpm to about 6050 rpm. In some instances, the shaking rate is 6000 rpm.

Similarly, certain enriched nano/microbubble populations may be formed by activating non-aqueous formulations at any of the foregoing shaking rates for a shaking time in the range of 40 to 100 seconds, or 45 to 95 seconds, or 46 to 94 seconds, or 47 to 93 seconds, or 48 to 92 seconds, or 49 to 91 seconds, or 40 to 100 seconds, including without limitation 40, 41, 42, 43, 44, 45, 46, 47, 48, 49, 50, 51, 52, 53, 54, 55, 60, 65, 70, 75, 80, 85, 86, 87, 88, 89, 90, 91, 92, 93, 94, 95, and 100 seconds. In other embodiments, the shaking time may be in the range of 70 to 95 seconds, including 71 to 94 seconds, 72 to 93 seconds, 73 to 92 seconds, 74 to 91 seconds, or 75, 76, 77, 78, 79, 80, 81, 82, 83, 84, 85, 86, 87, 88, 89 or 90 seconds. Any range defined by lower and upper boundaries selected from these times is contemplated by this disclosure.

Moreover, it was surprisingly found that when these non-aqueous lipid formulations, such as those described in Example 2, occupied about 9% of the internal volume of the container with gas occupying about 91% of the internal volume of the container (as is described in the Examples and denoted as DEFINITY RT 1.5× or DEFINITY RT 1×+0.5×) then the optimal shaking speed was about 6000 rpm and optimal shaking time was about 60 seconds. The shaking speed (or rate, as the terms are used interchangeably) could range from 6000 rpm+/−10% or +/−5%, or +/−1%, and/or about 5000 rpm to about 7000 rpm, or about 5500 rpm to about 6500 rpm, or about 5800 rpm to about 6200 rpm, or about 5900 to about 6100 rpm, or about 5950 rpm to about 6050 rpm. In some instances, the shaking rate is 6000 rpm. The shaking time could range from 55 to 65 seconds, 56 to 64 seconds, 57 to 63 seconds, 58 to 62 seconds, 59 to 61 seconds, or it could be 60 seconds. The compositions of DEFINITY and DEFINITY RT are provided in the Examples,

Even more surprisingly, these particular formulations activated according to these parameters generate populations wherein 27-56% of microbubbles had a diameter in the range of about 2 microns to less than or about 6 microns. Such enriched microbubble populations have not been previously reported without additional separation procedures.

Certain activation parameters resulted in enrichment of two microbubble subpopulations. Some of these nano/microbubble populations were enriched for microbubbles having a diameter in the range of about 2 to less than or about 6 microns and for microbubbles having a diameter in the range of about 6 microns to about 10 microns. For example, a shaking rate of about 6000 rpm for a shaking time of about 60 seconds applied to a non-aqueous lipid formulation, such as that described in Example 2, occupying about 9% of the internal volume of the container with gas occupying about 91% of the internal volume of the container (as described in the Examples) resulted in a population enriched for microbubbles having a diameter in the range of about 2 to about 6 microns and in the range of about 6 to about 10 microns. The shaking rate could range from 6000 rpm+/−10% or +/−5%, or +/−1%, and/or about 5000 rpm to about 7000 rpm, or about 5500 rpm to about 6500 rpm, or about 5800 rpm to about 6200 rpm, or about 5900 to about 6100 rpm, or about 5950 rpm to about 6050 rpm. In some instances, the shaking rate is 6000 rpm, or 6200 rpm, or 6600 rpm. The shaking time could range from 55 to 65 seconds, 56 to 64 seconds, 57 to 63 seconds, 58 to 62 seconds, 59 to 61 seconds, or it could be 60 seconds.

A shaking rate of about 6000 rpm for a shaking time of about 90 seconds applied to another non-aqueous lipid formulation, such as that described in Example 2, caused enrichment of the nanobubble population as well as enrichment of the microbubble population having a diameter in the range of about 2 microns to about 6 microns. The shaking rate could range from 6000 rpm+/−10% or +/−5%, or +/−1%, and/or about 5000 rpm to about 7000 rpm, or about 5500 rpm to about 6500 rpm, or about 5800 rpm to about 6200 rpm, or about 5900 to about 6100 rpm, or about 5950 rpm to about 6050 rpm, or about 6000 rpm to about 6600 rpm. In some instances, the shaking rate is 6000 rpm or 6200 rpm or 6600 rpm. The shaking time could range from 85 to 95 seconds, 86 to 94 seconds, 87 to 93 seconds, 88 to 92 seconds, 89 to 91 seconds, or it could be 90 seconds. As another example, it has been found that aqueous lipid formulations provided herein, such as those described in Example 1, also must be activated for longer periods of time or at higher speeds (or rates) to generate certain enriched nano/microbubble populations of this disclosure. For example, certain aqueous lipid formulations generate microbubbles for diagnostic ultrasound applications when activated at 4530 rpm for 45 seconds. However, in order to generate the nano/microbubble populations provided herein, these aqueous formulations must be activated at significantly higher rates, including at about 6000 rpm, for shorter times, ranging from about 20 to about 30 seconds. Activation of these lipid formulations at about 6000 rpm for about 30 seconds provides populations enriched in microbubbles having a diameter in the range of about 2 microns to about 6 microns. Activation of these lipid formulations at about 6000 rpm for about 20 to 25 seconds provides nanobubble-enriched populations. The shaking rate could range from 6000 rpm+/−10% or +/−5%, or +/−1%, and/or about 5000 rpm to about 7000 rpm, or about 5500 rpm to about 6500 rpm, or about 5800 rpm to about 6200 rpm, or about 5900 to about 6100 rpm, or about 5950 rpm to about 6050 rpm, or about 6000 rpm to about 6600 rpm. In some instances, the shaking rate is 6000 rpm or 6200 rpm or 6600 rpm. The shaking time could range from 15 to 35 seconds, 16 to 34 seconds, 17 to 33 seconds, 18 to 32 seconds, 19 to 31 seconds, or it could be 20, 21, 22, 23, 24, 25, 26, 27, 28, 29, or 30 seconds.

Alternatively, these same aqueous lipid formulations (Example 1), can be activated for significantly longer times using the lower rates (e.g., about 4530 rpm for about 90 seconds) in order to generate nanobubble-enriched populations. The shaking rate could range from 4930+/−10%, or +/−5%, or +/−1%, and/or about 4000 to about 5000 rpm, or about 4100 to about 4900 rpm, or about 4200 to about 4800 rpm, or about 4300 to about 4700 rpm, or about 4400 to about 4600 rpm, or about 4500 to about 4600 rpm, or about 4500 to about 4550 rpm, or about 4530 rpm. The shaking time could range from 85 to 95 seconds, or 86 to 94 seconds, or 87 to 93 seconds, or 88 to 92 seconds, or 89 to 91 seconds, or it could be 90 seconds.

These same aqueous lipid formulations (Example 1) can be activated at higher shaking rates for shorter times (e.g., about 6000 rpm for about 30 seconds) in order to generate populations enriched for microbubbles having a diameter in the range of about 2 microns to about 6 microns. The shaking rate could range from 6000 rpm+/−10% or +/−5%, or +/−1%, and/or about 5000 rpm to about 7000 rpm, or about 5500 rpm to about 6500 rpm, or about 5800 rpm to about 6200 rpm, or about 5900 to about 6100 rpm, or about 5950 rpm to about 6050 rpm, or about 6000 rpm to about 6600 rpm. In some instances, the shaking rate is 6000 rpm or 6200 rpm or 6600 rpm. The shaking time could range from 25 to 35 seconds, 26 to 34 seconds, 27 to 33 seconds, 28 to 32 seconds, 29 to 31 seconds, or it could be 30 seconds.

Some of the preparatory methods provided herein involve the use of a 2 mL vial as a container housing the lipid formulation and the gas. In some instances, the vial contains about 1.76 ml of an aqueous lipid formulation and about 2.04 ml of gas. Accordingly, the lipid formulation occupies about 46% of the total internal volume (i.e., volume of lipid formulation as a percentage of the total internal volume) and the gas occupies about 54% of the total internal volume (i.e., volume of gas as a percentage of the total internal volume).

Some of the preparatory methods provided herein involve the use of a 2 mL vial as a container housing a smaller volume of a non-aqueous lipid formulation as well as the gas. For example, the vial having a total internal volume of about 3.8 ml may contain about 0.35 ml of non-aqueous lipid formulation and about 3.45 ml of gas. Accordingly, the lipid formulation occupies about 9% of the total internal volume and the gas occupies about 91% of the total internal volume.

Another example involves a vial having a total internal volume of about 3.8 ml that contains about 0.52 ml of non-aqueous lipid formulation and about 3.28 ml of gas. Accordingly, the lipid formulation occupies about 14% of the total internal volume and the gas occupies about 86% of the total internal volume. Examples of these latter formulations are described in the Examples and denoted as DEFINITY-RT 1.5× and DEFINITY-RT 1×+0.5×. This lipid formulation to gas volume ratio (14% to 86%) surprisingly has been found to be useful when generating populations of bubbles enriched for microbubbles ranging in size from about 2 microns to about 10 microns, including about 2 microns to less than or about 6 microns, and about 6 microns to about 10 microns. Also surprisingly, this effect was apparently not dependent on lipid concentration since reducing the concentration by 33% (e.g., from about 3.75 mg/ml to about 2.5 mg/ml) had no impact provided the lipid formulation volume to gas volume remained constant.

Accordingly, depending on the enrichment desired, and in keeping with the teachings provided herein including the Examples, the lipid formulations provided herein may be activated in a number of ways and using a number of shaking rate/speed and shaking time combinations.

Aqueous lipid formulations, such as but not limited to the aqueous lipid formulation described in Example 1, may be activated at about 4530 rpm for about 10, about 20, about 30, about 40, about 50, about 60, about 70, about 75, about 80, or about 90 seconds.

Aqueous lipid formulations alternatively may be activated at about 6000 rpm for about 5-30 seconds including without limitation about 5, about 10, about 15, about 20, about 25, or about 30 seconds.

Non-aqueous lipid formulations, such as but not limited to the non-aqueous lipid formulation described in Example 2, may be activated at about 4950 rpm for about 10 to about 90 seconds including without limitation about 10, about 20, about 30, about 40, about 50, about 60, about 70, about 75, about 80, about 90 seconds, or about 120 seconds.

Non-aqueous lipid formulations, such as but not limited to the non-aqueous lipid formulation described in Example 2, may be activated at about 6000 rpm for about 10 to about 90 seconds including without limitation about 10, about 20, about 30, about 40, about 50, about 60, about 70, about 75, about 80, or about 90 seconds.

Non-aqueous lipid formulations, such as but not limited to the non-aqueous lipid formulation described in Example 2 and provided at a 1.5× fill volume (relative to the fill volume recited in Example 2) may be activated at about 6000 rpm for about 40 seconds, 45 seconds, 50 seconds, 55 seconds, 60 seconds, 65 seconds, 70 seconds, 75 seconds, or 80 seconds. In some instances, it is activated at about 6000 rpm for about 60 seconds.

Non-aqueous lipid formulations, such as but not limited to the non-aqueous lipid formulation described in Example 2 and provided at a 1× fill volume plus a 0.5× fill volume of lipid-less matrix (relative to the fill volume recited in Example 2) may be activated at about 6000 rpm for about 40 seconds, 45 seconds, 50 seconds, 55 seconds, 60 seconds, 65 seconds, 70 seconds, 75 seconds, or 80 seconds. In some instances, it is activated at about 6000 rpm for about 60 seconds.

Other embodiments of this disclosure involve the formation of nanodroplets, optionally starting from a population of gas nanobubbles and microbubbles. Nanodroplets, as used herein, are nanometer-sized (less than 500 nm, typically) droplets having a center occupied by gas in its liquid form and an outer lipid shell. One example of a gas that may be present in its liquid form is a perfluorocarbon gas such as but not limited to perfluoropropane. Other perfluorocarbon gases are contemplated, and examples are provided herein. Perfluoropropane has a boiling point of −36.7° C. yet can remain in liquid form at body temperatures and this can be exploited to create nanodroplets that may travel through the body more efficiently than larger gas bubbles.

In some embodiments, the nanodroplets are formed by condensing microbubbles. As an example, lipid encapsulated gas microbubbles ranging in size from 0.5-500 microns may be readily condensed to form nanodroplets. This may be accomplished by placing the microbubble population in a syringe, such as a 3 mL syringe, and applying hand pressure on a plunger. It was found that the microbubble suspension changed from a milky white appearance to a clear colorless appearance upon application of such hand pressure, leading to the conclusion that the milky white appearance correlated with the presence of microbubbles having a size in the range of 0.5-500 microns and that the clear, colorless appearance correlated with a significant decrease in such microbubbles and the condensation of gas to its liquid form. The nanodroplets may be formed without changing the temperature of the microbubble population (e.g., without decreasing the temperature).

The nanodroplets may comprise a shell of one or more lipids, such as phospholipids. The one or more phospholipids may be selected from DPPA, DPPC and DPPE, although they are not so limited. As with other embodiments in this disclosure one or more of these phospholipids may be PEGylated. In some embodiments, the DPPE is present in both PEGylated and un-PEGylated forms. Accordingly, some nanodroplet populations may have a lipid shell made of DPPA, DPPC and DPPE, optionally where the DPPE is PEGylated (e.g., including but not limited to PEG1000-DPPE, MPEG1000-DPPE, PEG2000-DPPE, MPEG2000-DPPE, PEG3000-DPPE, MPEG3000-DPPE, PEG5000-DPPE or MPEG5000-DPPE, and other examples of PEGylated DPPE provided herein). Other nanodroplet populations may have a lipid shell made of DPPC, DPPE and PEGylated DPPE, and may lack DPPA.

The manual (or hand) pressure to be applied to the microbubble preparation present in a syringe such as a 3 mL syringe may be in the range of 6-12 atm. A sufficient manual pressure will be achieved and will be apparent by the conversion of the milky white appearance of the preparation into a clear, colorless appearance.

The nanodroplets have a size of about less than 500 nm, including in the range of about 50 nm to about 450 nm, about 50 nm to about 400 nm, about 50 nm to about 350 nm, about 50 nm to about 300 nm, about 50 nm to about 200 nm, or about 100 nm to about 200 nm. Given the ability of the nanodroplets to access sites in the body which are inaccessible to the larger microbubbles, the nanodroplets may be used for a wider or different variety of in vivo applications as compared to microbubbles. Accordingly, this disclosure contemplates and provides methods that involve administering such nanodroplets to a subject for diagnostic or therapeutic benefit. As an example, a nanodroplet preparation may be administered to a subject and then may be induced to expand by converting its liquid center to a gaseous center. In doing so, the nanodroplet converts into a nano gas bubble and it may cause expansion in the tissue or vasculature where it is present.

These nanodroplets are contemplated for use in any of the diagnostic and therapeutic uses provided herein.

Reference may be made to Choudhary et al., J American Society Echocardiography, 30 (2): 189-197, 2017; and Hayward et al., Scandanavian J Rheumatology, 40 (5): 379-382, 2011.

### Therapeutic Applications

In addition to the contemplated use of certain nano/microbubble and nanodroplet populations of this disclosure for enhanced contrast imaging, this disclosure also contemplates use of these nano/microbubble and nanodroplet populations in therapeutic applications. These populations may be used in vivo in human or non-human subjects or in vitro. The nano/microbubble and nanodroplet populations provided herein therefore may be used for imaging purposes, therapeutic purposes, or for combined diagnostic and therapeutic purposes.

The lipid formulations may be activated as described herein in order to form a sufficient number of nano/microbubbles, which in turn are optionally diluted into a larger volume, and administered in one or more bolus injections or by a continuous infusion. Administration is typically intravenous injection. Alternatively, the activated formulation may be subjected to pressure to form nanodroplets, as described herein, and the nanodroplets may be administered, optionally in one or more bolus injections or by a continuous infusion. Therapeutic ultrasound is then performed concurrently with administration or shortly thereafter. The ultrasound can be directed to any region of the body including but not limited to heart, blood vessels, the cardiovasculature, the liver, the kidneys, the penis, and the head.

Subjects of the invention include but are not limited to humans and animals. Humans are preferred in some instances. Animals include companion animals such as dogs and cats, and agricultural or prize animals such as but not limited to bulls and horses.

The nano/microbubble and nanodroplet populations are administered in effective amounts. An effective amount will be that amount that facilitates or brings about the intended in vivo response and/or application. The therapeutic methods provided herein are intended to treat, partially or completely, a condition.

This disclosure further contemplates use of the enriched nano/microbubble or nanodroplet populations provided herein together with ultrasound in therapeutic applications. Ultrasound has previously been used or contemplated for use in therapeutic applications. Combining ultrasound with nano/microbubbles or nanodroplets, as provided herein, may provide additional benefit, potentially with less adverse effects of the ultrasound.

When used alone, ultrasound is hypothesized to have a number of different mechanisms of action, including for example heating of target tissues, mechanical disruption, and the like. The heating of tissues, in some embodiments, may be an undesirable effect.

Gas-filled nano/microbubbles or nanodroplets used together with ultrasound may also have a number of mechanisms of action. One particular mechanism of action is cavitation. Cavitation occurs when administered nano/microbubbles or nanodroplets absorb the ultrasound energy and in turn undergo a number of mechanical changes. In some instances, the energy absorbed by the nano/microbubbles or nanodroplets is released from the nano/microbubbles or nanodroplets, and then absorbed by the surrounding tissue. In the case of stable cavitation, the nano/microbubbles such as microspheres or the nanodroplets may undergo one or more cycles of swelling and contracting, without ultimate rupturing. In the case of inertial cavitation, the nano/microbubbles such as microspheres or the nanodroplets rupture. Whether stable or inertial cavitation occurs depends on a number of factors including the energy level of the ultrasound, the location of intended use, and the nature or composition of the nano/microbubbles or nanodroplets.

For some therapeutic applications, stable cavitation is sufficient to effect the desired clinical benefit. For other therapeutic applications, inertial cavitation is required to effect the desired clinical benefit. As an example, if the nano/microbubbles such as microspheres or nanodroplets are used as delivery vehicles, then for maximum effect the they should rupture in order to release their cargo in vivo.

The ultrasound energy may be applied externally or internally. Externally applied ultrasound is preferable in most situations because it is less (if at all) invasive. Internally applied ultrasound may be used in some instances where the target tissue is a deep tissue and/or externally applied ultrasound might cause adverse effects in intervening tissue. Ultrasound may be applied externally by placing an ultrasound transducer in close proximity to an outer surface of a subject, such as the skin or an outwardly facing cavity such as the oral cavity, etc. Ultrasound may be applied internally by introducing an ultrasound transducer into the body of a subject, typically on a probe. An example may be a cardiovascular probe capable of traveling through the circulatory system.

Conditions that could benefit from the combined use of nano/microbubbles or nanodroplets and ultrasound energy are described below. Typical ultrasound frequencies are in the range of about 1-6 MHz for deeper tissues such as the kidney and heart and 7-10 MHz for superficial tissues such as the carotid artery and breast.

Sonothrombolysis refers to the dissolution or degradation of a thrombus using ultrasound. The efficacy of ultrasound can be enhanced by combining it with the nano/microbubbles or nanodroplets of this disclosure. Thromboembolic diseases that would benefit from sonothrombolysis include acute myocardial infarction (e.g., acute ST-elevation myocardial infarction), pulmonary thromboembolism, deep vein thrombosis, retinal vein occlusion (together with ophthalmic ultrasound), peripheral artery disease, cardiac atherosclerosis, microvascular obstruction or thrombosis, and stroke. The combined therapy provided herein may also be used in the treatment of kidney stones.

Patients experiencing a thromboembolic event may be treated with a combination of ultrasound, nano/microbubbles or nanodroplets, and a therapeutic agent such as urokinase or t-PA.

The nano/microbubbles or nanodroplets used for these indications may be targeted to a particular region of the body or a particular structure, such as for example a thrombus. As an example, the nano/microbubbles or nanodroplets may be targeted to thrombi by conjugation to an Arg-Gly-Asp (RGD) peptide that binds specifically to platelet glycoprotein IIb/IIIa. See Ma et al. Bioconjugate Chemistry, 2020, 31:369-374 for a description of the labeling methodology. The nanobubble enriched populations as well as the microbubble enriched populations provided herein as well as the nanodroplets provided herein may be used in this therapeutic application. The ultrasound frequency may be about 1.3 MHz. The method may be carried out with an ultrasound treatment probe C5-1s.

Sonoporation refers to the use of ultrasound to increase permeability of cell membranes, vasculature, and the blood-brain-barrier (BBB), typically in a reversible manner. By co-administering nano/microbubbles or nanodroplets with a therapeutic agent, or administration of nano/microbubbles or nanodroplets conjugated to a therapeutic agent and applying ultrasound, it is possible to increase the permeability of a membrane, barrier, or vasculature in order to deliver the therapeutic agent to specific regions of the body. Conditions that would benefit from this combined approach include cardiovascular disease, solid tumors such as glioblastomas, and neurodegenerative diseases such as Alzheimer's disease and Parkinson's disease. Both nano- and micro-bubbles and nanodroplets would be suitable for these applications. Nanobubbles and nanodroplets may have the added advantage of being capable of passively extravasating into tissues such as tumors from the circulation as a function of their smaller size. Additionally, tumors typically have leaky vasculatures. Nanobubbles and nanodroplets may be used at off-resonant frequencies such as 1-10 MHz. Sonoporation can also be used to sensitize hypoxic tumors to radiation therapy.

Drug delivery approaches are also contemplated by this disclosure. As mentioned above, therapeutic agents may be administered before, during or after the administration of the nano/microbubble or nanodroplet populations and application of ultrasound. These agents may be separate from the nano/microbubbles or the nanodroplets or they may be carried in the internal cavity of the nano/microbubble or nanodroplet, or they may be attached to the surface of the nano/microbubble or nanodroplet, or they may be embedded in the lipid shell of the nano/microbubble or nanodroplet. Accordingly, some of these drug delivery applications may be targeted applications and some may be untargeted. Agents to be delivered include nucleic acids, proteins, cells, cell fragments such as exosomes.

Still other applications include delivery of oxygen to tumors in order to render such tumors sensitive to radiation treatment.

Still other applications include connective tissue disorders, such as but not limited to Peyronie's disease, as well as tendinitis, bursitis. Other conditions to be treated include uterine fibroid ablation, cataract removal, surgical tissue cutting and hemostasis, bone fracture healing, Meniere's disease (by destruction of the vestibular nerve), glaucoma, laparoscopic tissue ablation. The combined use of gas bubbles or droplets such as the nanodroplets provided herein and ultrasound may be used as part of a neurosurgery, ultrasound assisted immunotherapy, ultrasound assisted neuromodulation.

Other conditions characterized by reduced blood flow in large or small vessel or reduced tissue perfusion would also benefit from combined use of the enriched gas bubble populations or nanodroplets provided herein and ultrasound. Examples include retinal vein occlusion, coronary artery occlusion, cardiac atherosclerosis, peripheral artery disease (and concomitant limb salvage) and other conditions benefiting from peripheral blood flow enhancement (e.g., sickle cell disease), atherosclerosis including removal or destruction of atherosclerotic plaques, arteriosclerosis, erectile dysfunction, stenosis and restenosis for example following balloon angiography. These methods can also be used to maintain vascular access for example for long term dialysis treatment, particularly involving the creation and/or maintenance of an arteriovenous fistula in the cephalic vein, including for example a brachiocephalic fistula (e.g., removal or destruction of clots, thrombi, stenosis, and the like).

The gas bubble or droplet including nanodroplet populations provided herein may also be used to enhance high intensity focused ultrasound (HiFu).

Imaging applications include deep tissue imaging such as but not limited to cardiac imaging, kidney imaging, liver imaging. Imaging applications also include breast imaging, imaging of any tissue or region of the body suspected of having a growth such as a tumor. These imaging applications can also involve enhancement of Doppler ultrasound imaging.

Clinical ultrasound parameters and suitable systems are known in the art. Examples include Philips EPIQ, Philips Sonos 7500, EKOS System (EKOS Endo Wave Infusion Catheter System, EKOS Corporation), an approved ultrasound thrombolytic device.

### Erectile Dysfunction

In still another aspect, the present disclosure provides methods of using a combination of ultrasound and gas bubbles or droplets such as the nanodroplets provided herein to treat erectile dysfunction (ED) including vasculogenic ED. The gas bubbles may be the enriched populations provided herein or they may be microbubble populations used for diagnostic ultrasound such as those present in activated DEFINITY (i.e., perflutren lipid microspheres having an average diameter range of 1.1 microns to 3.3 microns, wherein 98% of the bubbles are less than 10 microns in diameter).

Erectile dysfunction (ED) is the inability to achieve or maintain an erection for satisfactory sexual performance at least some of the time (Yafi, et al, Nat. Rev. Disease Primers, 2, Article number: 16003 (2016)). ED is positively correlated with age; the same study found that 6.5 percent of men ages 20-29 reported experience with ED, whereas 77.5 percent of men aged over 75 years of age reported experience with ED.

ED occurs to differing degrees in affected individuals; the severity may be assessed with the International Index of Erectile Function (IIEF-5), which is a five-item questionnaire (Rhoden, International Journal of Impotence Research, 14, 245-250 (2002)). IIEF-5 scores of 1-7 indicate severe ED, 8-11 indicates moderate ED, 12-16 indicates mild-to-moderate ED, 17-21 indicates mild ED, and 22-25 indicates no ED. Severity may also be assessed using the IIEF-15, which is a 15 item questionnaire. IIEF-15 scores of 1-10 indicate severe ED, 11-16 indicates moderate ED, 17-21 indicates mild to moderate ED, 22-25 indicates mild ED, and 26-30 indicates no ED.

Physiological causes of ED are divided into endocrine and nonendocrine causes. Endocrine causes relate to hormonal issues, particularly reduced serum testosterone. The most common nonendocrine cause is vasculogenic, which are disorders relating to blood supply.

ED in subjects having vasculogenic risk factors (including but not limited to, coronary artery disease (CAD), hypertension, dyslipidemia, smoking, and diabetes mellitus) and not having risk factors for psychogenic, urogenital, endocrinological, or neurological causes of ED are considered to have vasculogenic ED (Yavuzgil, et al, Int. J. Cardiol. (2005) 103:19-26). Blood flow can also be assessed by penile doppler ultrasound (Koca, et al, J Sex Med. (2010) 7 (12): 3997-4002).

The combination bubble/ultrasound therapy may be supplemented with the administration of therapeutic agents used to treat ED. One common pharmaceutical treatment, PDE5 inhibitors, such as sildenafil, vardenafil, tadalafil, and avanafil, inhibit PDE5, allowing a buildup of the signals that promote erection of the penis. PDE5 inhibitors are effective, but may interfere with other medications (such as nitrate-containing medications). When used with the bubble/ultrasound combination therapy, it may be possible to use a sub-therapeutic dose of these PDE5 inhibitors.

Thus, in one aspect, the present disclosure provides methods of treating ED in a subject in need thereof. In some embodiments, subjects are identified based upon a score on the IIEF-5 or IIEF-15 questionnaire. In an embodiment, prior to treatment subjects receive or have received a score indicating severe ED (e.g., 1-7 for IIEF-5 or 1-10 for IIEF-15). In some embodiments, the subjects have received a score indicating severe or moderate ED (e.g., 11 or lower for IIEF-5 or 16 or lower for IEF-15). In some embodiments, the subjects have received a score indicating severe, moderate, or mild-to-moderate ED (e.g., 16 or lower for IIEF-5 or 21 or lower for IIEF-15). In some embodiments, the subjects have received a score indicating at least mild ED (e.g., 21 or lower for IIEF-5 or 25 or lower for IIEF-15).

In some embodiments, the subjects to be treated are those characterized by either test to have mild to moderate ED. Treatment may be measured by an improved IIEF-5 or IIEF-15 score. A subject who is treated according to this aspect has a score that increases by 1, 2, 3, 4, 5, 6, 7, 8, 9, 10, 11, 12 or 13 points following treatment, according to the IIEF-15 test. Increased scores on the IIEF-5 test similarly will evidence treatment of the subject.

In some embodiments, the subject receives a higher IIEF-5 score after treatment (indicating less-severe ED). In some embodiments, the subject receives an IIEF-5 score 3 point higher, 4 points higher, 5 points higher, 6 points higher, 7 points higher, 8 points higher, 9 points higher, 10 points higher, or more than 10 points higher.

The subject is then administered the gas bubble populations such as those present in activated DEFINITY, or any of the enriched populations described herein, or the nanodroplets provided herein. These bubble or droplet preparations may be administered as one or more bolus doses or as a continuous infusion. Intravenous administration is preferred.

The subject may be subjected to localized (or targeted) ultrasound in the genital area, including in the corpora cavernosa, pundendal or bulbospongiosis regions.

The ultrasound may be applied during the infusion of the bubbles or droplets, or following the infusion of the bubbles or droplets including but not limited to 2-20 minutes, 2-10 minutes, 5-10 minutes, 5-15 minutes, or 5-20 minutes thereafter. In some embodiments, DEFINITY microbubbles are used because they may remain in circulation longer than other microbubble formulations.

In an embodiment, the subject is treated once. In an embodiment, the subject is treated twice. In an embodiment, the subject is treated at least 3 times. The subject may undergo a plurality of sessions (e.g., 4-8 sessions) at spaced intervals, optionally regularly spaced intervals. The treatment may last for 1 or more weeks or 1 or more months. The subject may be re-treated every 1, 2, 3, 4, 5, 6, 7, 8, 9, 10, 11 or 12 months. High energy (or high intensity) ultrasound may be used. Ultrasound energy may be applied using a broad-band pulse centered at 2.0 MHz at a mechanical index of 1.0, using for example a phased array transducer interfaced with an ultrasound system such as the iE33 Philips ultrasound system. In other embodiments, ultrasound is performed over the genital area and optionally specifically the corpora cavernosa at 1.3 MHZ with a mechanical index of 1.3 and a pulsing interval of 3 seconds. In still another embodiment, ultrasound may be performed using harmonic power Doppler (Sonos 5500, Philips Ultrasound) at 1.3 MHz with a pulse repetition frequency of 9.3 kHz, a mechanical index of 1.3, and the acoustic focus set at the level of the corpora cavernosa. Other variations are contemplated and may be determined by the skilled person.

In some embodiments, subjects are further identified by presence of vasculogenic risk factors. In some embodiments, subjects have coronary artery disease (CAD), hypertension, dyslipidemia, smoking, or diabetes mellitus. In some embodiments, subjects have two or more of the disorders selected from: coronary artery disease (CAD), hypertension, dyslipidemia, smoking, and diabetes mellitus.

### Activation, Generally

Activation may be achieved by vigorous shaking of the lipid formulation in the presence of a gas. Vigorous shaking, as used herein, refers to a motion that agitates a formulation, whether aqueous or non-aqueous, such that gas is introduced from the local ambient environment within the container (e.g., vial) into the formulation. Any type of motion that agitates the formulation and results in the introduction of gas may be used for the shaking. The shaking must be of sufficient force to allow the formation of foam after a period of time.

Activation is typically performed just before administration to a subject and therefore is performed by an end user or an intermediate, but not the supplier or manufacturer of the lipid formulation. The lipid formulations are typically provided in vials that minimally house the lipid formulation and a gas in the headspace. The vigorous shaking of the lipid formulation in the presence of the gas results in the formation of lipid-encapsulated gas-filled nano/microbubbles that can be used in therapeutic applications. By “gas-filled”, as used herein, it is meant the nano/microbubbles comprise gas, such as a perfluorocarbon gas including but not limited to perflutren (or perfluoropropane) gas, in their internal cavity. The lipid shell that encapsulates the gas may be arranged as a unilayer or a bilayer, including unilamellar or multilamellar bilayers.

The lipid formulations are typically provided in containers such as vials with a headspace gas of interest. The container may be made of any material including but not limited to glass or plastic. The glass may be pharmaceutical grade glass. The container may be sealed with a stopper such as a rubber stopper.

As will be understood in the context of this disclosure, the internal volume of a container may be occupied with a lipid formulation and gas. Containers may be defined based on their liquid fill volume, which refers to the volume of liquid (e.g., lipid formulation) typically placed into the container. Liquid fill volume is different from and typically lower than the entire internal volume of the container. When containers are defined by a volume measurement herein, it is to be understood that this refers to the liquid fill volume. For example, a 0.5-10 mL container is a container having a liquid fill volume of 0.5-10 mL. Examples of suitable containers and their corresponding liquid fill and internal volumes are as follows: Schott 2 mL (liquid fill volume) vial having a 2.9 mL internal volume; Schott 3 mL (liquid fill volume) vial having a 4.5 mL internal volume; and Wheaton 1 mL (liquid fill volume) v-vial having a 1.2 mL internal volume.

In some embodiments, the container is a 0.5-10 mL container, such as 1-5 mL container, or a 1 mL or 2 mL container. The container may be a vial such as a glass vial. An example of a suitable vial is the Wheaton 2 ml glass vial (commercially available from, for example, Nipro, Cat. No. 2702, B33BA, 2 cc, 13 mm, Type I, flint tubing vial), having an actual internal volume of about 3.8 ml.

The vial may be pre-filled with gas of interest such as perflutren gas, prior to the introduction of the lipid formulation, or lipid formulation may be introduced into the vial first followed by the gas (e.g., through a process referred to as headspace gas exchange). The containers are preferably sterile and/or are sterilized after introduction of the lipid formulation and/or gas as described in published PCT application WO99/36104.

In some embodiments, the container is a flat bottom container such as a flat-bottom vial. Suitable vials include flat bottom borosilicate vials, including Wheaton vials. In some embodiments, the container is a non-flat bottom container or vial. In some embodiments, the container is a V-bottom container such as a V-bottom vial. In some embodiments, the container is a round-bottom container such as round-bottom vial. In some embodiments, the container has converging walls such that its bottom surface area (or bottom surface diameter) is smaller than its top (opening) surface area (or diameter) or smaller than any diameter therebetween (e.g., a body diameter). For clarity, a V-bottom container or vial has converging walls, and its bottom surface area is significantly smaller than any of it top or body surface areas.

In some embodiments, the container is a syringe. The lipid formulation may be provided in a pre-filled syringe, optionally in physical contact with the gas. In some embodiments, the container is a single chamber container, such as a vial.

Activation may produce at least 1×10⁷ microspheres per ml of lipid formulation, at least 5×10⁷ microspheres per ml of lipid formulation, at least 7.5×10⁷ microspheres per ml of lipid formulation, at least 1×10⁸ microspheres per ml of lipid formulation, at least 1×10⁹ microspheres per ml of lipid formulation, or at least 5×10⁹ microspheres per ml of lipid formulation, at least 10¹⁰ microspheres per ml of lipid formulation, or at least 1.5×10¹⁰ microspheres per ml of lipid formulation.

The shaking may be by swirling (such as by vortexing), side-to-side, or up and down motion. Further, different types of motion may be combined. The shaking may occur by shaking the container (e.g., the vial) holding the aqueous or non-aqueous lipid solution, or by shaking the aqueous or non-aqueous solution within the container (e.g., the vial) without shaking the container (e.g., the vial) itself. Shaking is carried out by machine in order to standardize the process. Mechanical shakers are known in the art and their shaking mechanisms or means may be used in the devices of the present disclosure. Examples include amalgamators such as those used for dental applications. Shaking encompasses at least 4000, at least 4500, at least 5000, at least 5500, at least 6000, at least 6100, at least 6200, at least 6300, at least 6400, at least 6500, at least 6600 or more shaking motions per minute.

The formation of gas-filled microspheres upon activation can be detected by the presence of a foam on the top of the aqueous or non-aqueous solution and the solution becoming white.

Activation is carried out at a temperature below the gel state to liquid crystalline state phase transition temperature of the lipid employed. By “gel state to liquid crystalline state phase transition temperature”, it is meant the temperature at which a lipid layer (such as a lipid monolayer or bilayer) will convert from a gel state to a liquid crystalline state. This transition is described for example in Chapman et al., J. Biol. Chem. 1974 249, 2512-2521. The gel state to liquid crystalline state phase transition temperatures of various lipids will be readily apparent to those skilled in the art and are described, for example, in Gregoriadis, ed., Liposome Technology, Vol. I, 1-18 (CRC Press, 1984) and Derek Marsh, CRC Handbook of Lipid Bilayers (CRC Press, Boca Raton, Fla. 1990), at p. 139.

It will be understood by one skilled in the art, in view of the present disclosure, that the lipid(s) or lipid microspheres may be manipulated prior to or subsequent to being subjected to the methods provided herein. For example, after the shaking is completed, the gas-filled bubbles may be extracted from their container (e.g., vial). Extraction may be accomplished by inserting a needle of a syringe or a needle-free spike (i.e., PINSYNC) into the container, including into the foam if appropriate, and drawing a pre-determined amount of liquid into the barrel of the syringe by withdrawing the plunger or by adding an aqueous liquid mixing and drawing a pre-determined amount of liquid into the barrel of the syringe by withdrawing the plunger.

Following activation, the nano/microbubbles may be diluted with saline, either in their container or following withdrawal from the container.

Microsphere diameter is typically measured using instrumentation known and available in the art including but not limited to a Malvern FPIA-3000 Sysmex particle sizer and Accusizer 780.

### Devices

Also provided herein are methods and means (e.g., devices) for facilitating proper and accurate preparation of the nano/microbubble and nanodroplet populations provided herein. These methods and means reduce the risk of improper preparation of such populations. Nano/microbubble or nanodroplet populations that are not properly and accurately prepared can result in various issues including for example having too few gas-filled nano/microbubbles or nandroplets, thereby reducing therapeutic efficacy. Proper identification and thus proper activation of a lipid formulation may be achieved using devices capable of uniquely identifying and uniquely activating diverse lipid formulations.

Identification of a formulation and/or distinction between different formulations can be achieved in a number of ways. For example, devices may be used with scanners able to read labels on the formulation container (e.g., vial). In other instances, identification and/or distinction between different formulations can be achieved using devices that recognize the shape and size of a container housing a formulation. These latter devices may comprise a single holder or they may comprise two or more holders.

According to one aspect, a device receives a container holding a formulation, identifies the formulation and performs different actions depending on the identity of the formulation that is detected. The device associates certain actions with each formulation, for example with reference to an internal or cloud-based look up table. After identifying a certain formulation, the device performs the actions required to activate that formulation, preferably in a fully automatic manner.

A variety of different actions can be performed based on the formulation that is detected. In some embodiments, the device shakes the vial and correspondingly the formulation. In some embodiments, the device performs a specific shaking duration, pattern, and/or rate depending on the formulation that is detected. Examples of different shaking patterns include but are not limited to: side to side reciprocation, up and down reciprocation, vibration, a spinning motion, a figure-eight path, a circular path and back-and-forth tilting (e.g., rotating the container by some angle and reversing the action), For example, in one illustrative embodiment, the device associates a shaking duration of about 60 seconds with formulation type “A” and about 90 seconds with formulation type “B.” When the device detects a formulation type “A,” the device automatically shakes the formulation for about 60 seconds without requiring the user to input a shaking time. When the device detects a formulation type “B,” the device automatically shakes the formulation for about 90 seconds. It should be appreciated that other actions can be associated with an identified and detected formulation.

In some embodiments, the container housing the lipid formulation includes an indicator that indicates the formulation type and the device includes a detector that reads the indicator to detect the formulation type. The indicator may be one that is machine—or device readable. Examples of machine—or device-readable indicators include magnetic stripes, chips/microchips, barcodes including linear, matrix and 2D barcodes, radio frequency identification (RFID) tags, color labels that are identifiable by color detection, and the like. Barcodes such as linear barcodes may be those that comply with or meet Uniform Code Council standards or Health Industry Business Communications Council standards. Such indicators may in turn be read, for example, from a device such as a magnetic stripe reader, a chip reader, a barcode scanner or reader, an RFID tag reader, and the like. Virtually any labeling technology that has been used for authentication and/or “track and trace” purposes may be used in conjunction with the containers provided herein.

The indicator may be positioned on any suitable portion of the sample container, such as the body of the container or the cap. In some embodiments, the indicator is integrally formed with or otherwise a part of the sample container. For example, the indicator may be a colored cap or a physical feature such as a protrusion or an indentation on the sample container. In other embodiments, the indicator is attached to the container via, for example, adhesive, magnets, book-and-loop type fasteners, mechanical arrangement such as sliding the indicator behind holding tabs, or any other suitable attachment arrangement.

The indicator may provide the end user or an intermediate handler of the container a variety of information including but not limited to source and/or producer of the formulation contained therein, including for example the name of the company or company subsidiary that made the formulation and/or that produced components of the formulation, the date on which the formulation was made, the physical location where the formulation was made, the date of shipment of the container, the treatment of the container including for example whether it was stored in a remote location and the conditions and length of such storage, the date on which the container was delivered, the means of delivery, the National Drug Code (NDC) as prescribed by the FDA, content of the container, dose and method of use including route of administration, etc. The indicator may serve one or more purposes including for example authentication of the container and the formulation contained therein. Authentication means the ability to identify or mark the container as originating and having been made by an authorized party, and it allows an end user or other party to identify container and formulations originating from another, unauthorized party. The indicator may also be used to track and trace a container. This feature can be used to follow a container and the formulation contained therein following production and up to the point of administration to a subject. In this regard, the movement of the container during that period of time may be stored in a database, and optionally such a database may be accessible to an end user to ensure the integrity of the formulation.

The indicator may also be a combined indicator, intending that it may contain information that is read using two different modes. For example, the indicator may contain information that is apparent and understandable to the visible eye (e.g., it may recite the name of the producer in words) and other information that is machine-readable, such as RFID embedded or barcode embedded information.

The indicator may also be a dual use indicator, intending that it may serve two or more purposes. For example, the indicator may contain information that identifies the formulation and further information that identifies the manufacturer and/or date of manufacture. This information may be conveyed in the same format or using different format (e.g., one may be provided in an RFID indicator and the other may be provided in a barcode label).

According to some embodiments, the identification and activation device includes a bolder arranged to hold a container comprising a lipid formulation and gas, and a shaking means arranged to shake the holder at least one speed. The shaking means includes a motor arranged to drive movement of the holder via a transmission, wherein at least a portion of the transmission is arranged to prevent slippage during an activation of the UCA formulation.

The transmission may be arranged to create friction and/or resistance to movement. The transmission may include at least one of a tooth belt, a drive chain, a O-ring, and a gear box. The transmission may include a gear having first and second toothed wheels, the first toothed wheel being attachable to the holder and the second toothed wheel being attachable to the motor.

In such embodiments, a tooth belt is arranged to engage with each of the first and second toothed wheels. The first toothed wheel may be positioned around a portion of a spindle attachable to the holder, and the second toothed wheel may be positioned around a portion of the motor, such as a shaft. The first and second toothed wheels may be substantially parallel to one another. The first and second toothed wheels also may be substantially co-planar with one another.

The transmission may also include first and second O-rings, wherein the first O-ring is positioned on a first lateral side of the first toothed wheel and the second O-ring is positioned on a second lateral side of the first toothed wheel. In some embodiments, each of the first and second O-rings and the first toothed wheel are positioned around the spindle.

The transmission may include first and second profiled wheels, such as sprockets, with teeth or cogs. As with the above, the first wheel may be attachable to the holder and the second wheel may be attachable to the motor. In some embodiments, the first and second wheels engage with a chain, track, or another material that is perforated or indented. For example, the teeth are engageable with the chain, track, or other material with the perforations or indentations. Another suitable identification and activation device includes a holder arranged to hold a container comprising a lipid formulation, an identification means arranged to identify the lipid formulation in the container; and a shaking means arranged to shake the holder at least one speed, wherein the shaking means includes a motor arranged to drive movement of the holder via a transmission, wherein the transmission includes at least one of a toothed, perforated, indented belt or track, a drive chain, and a gear box.

The transmission may include a gear having first and second toothed wheels, the first toothed wheel being attachable to the holder and the second toothed wheel being attachable to the motor. The toothed, perforated, indented belt or track may be arranged to engage with each of the first and second toothed wheels. The transmission may include first and second profiled wheels, such as sprockets, that are attachable to the motor and holder, respectively. The toothed, perforated, indented belt or track or other material (e.g., a drive chain) may be arranged to engage with each of the first and second wheels.

Each of the foregoing apparati or devices may further include a housing, the shaking means being disposed in the housing. They also may include a first identification means arranged to identify the lipid formulation in the vial. The first identification means may include at least one of an RFID reader, a microchip reader, and a barcode scanner. The first identification means also may include at least one antenna.

The holder may include a shaker arm. The motor may include a direct current (DC) motor, such as a brushless DC motor.

The at least one speed may be pre-determined. The at least one speed may be pre-set by the apparatus. The at least one speed may be less than about 4530 rpm or it may be about 6000 rpm. The apparatus may be arranged to shake the holder at two or more speeds, wherein the two or more speeds are between about 4530 rpm and about 6000 rpm.

The identification means may be further arranged to identify a shaking speed for the lipid formulation in the container. The identification means may be arranged to identify the formulation and/or the shaking speed for the formulation by reading an indicator on the container.

### Lipids

The lipid formulations provided herein may comprise one and typically more than one lipid. As used herein, “lipids” or “total lipid” or “combined lipids” means a mixture of lipids. The lipids may be provided in their individual solid state (e.g., powdered) forms. Alternatively, the lipids may be provided as a lipid blend. Methods of making a lipid blend include those described in U.S. Pat. No. 8,084,056 and published PCT application WO 99/36104. A lipid blend, as used herein, is intended to represent two or more lipids which have been blended resulting in a more homogeneous lipid mixture than might otherwise be attainable by simple mixing of lipids in their individual powdered form. The lipid blend is generally in a powder form. A lipid blend may be made through an aqueous suspension-lyophilization process or an organic solvent dissolution-precipitation process using organic solvents. In the aqueous suspension-lyophilization process, the desired lipids are suspended in water at an elevated temperature and then concentrated by lyophilization. The contents of U.S. Pat. No. 8,084,056 and published PCT application WO 99/36104 relating to the method of generating a lipid blend are incorporated by reference herein.

Alternatively, the lipids may be provided as individual powders that are dissolved together or individually directly into propylene glycol, glycerol or propylene glycol/glycerol to form the non-aqueous lipid formulation or to form a precursor to the aqueous lipid formulation. The lipids may be cationic, anionic or neutral lipids. The lipids may be of either natural, synthetic or semi-synthetic origin, including for example, fatty acids, fluorinated lipids, neutral fats, phosphatides, oils, fluorinated oils, glycolipids, surface active agents (surfactants and fluorosurfactants), aliphatic alcohols, waxes, terpenes and steroids.

At least one of the lipids may be a phospholipid. A phospholipid, as used herein, is a fatty substance containing an oily (hydrophobic) hydrocarbon chain(s) with a polar (hydrophilic) phosphoric head group. Phospholipids are amphiphilic.

Preferably all of the lipids are phospholipids, examples of which include but are not limited to phosphatidylcholine; 1,2-dipalmitoyl-sn-glycero-3-phosphatidylcholine (DPPC); phosphatidic acid; 1,2-dipalmitoyl-sn-glycero-3-phosphatidic acid (DPPA); phosphatidylethanolamine; and 1,2-dipalmitoyl-sn-glycero-3-phosphatidylethanolamine (DPPE). DPPA and DPPE may be provided as monosodium salt forms.

In some instances, one or more of the lipids may be modified. For example, lipids may be conjugated to polymers, such as chitin, hyaluronic acid, polyvinylpyrrolidone, or polyethylene glycol (PEG). Lipids conjugated to PEG are referred to herein as PEGylated lipids. Preferably, the PEGylated lipid is DPPE-PEG or DSPE-PEG.

Conjugation of the lipid to the polymer such as PEG may be accomplished by a variety of bonds or linkages such as but not limited to amide, carbamate, amine, ester, ether, thioether, thioamide, and disulfide (thioester) linkages.

Terminal groups on the PEG may be, but are not limited to, hydroxy-PEG (HO-PEG) (or a reactive derivative thereof), carboxy-PEG (COOH-PEG), methoxy-PEG (MPEG), or another lower alkyl group, e.g., as in iso-propoxyPEG or t-butoxyPEG, amino PEG (NH2PEG) or thiol (SH-PEG).

The molecular weight of PEG may vary from about 500 to about 10000, including from about 1000 to about 7500, and from about 1000 to about 5000. In some important embodiments, the molecular weight of PEG is about 5000. Accordingly, DPPE-PEG5000 or DSPE-PEG5000 refers to DPPE or DSPE having attached thereto a PEG polymer having a molecular weight of about 5000.

The percentage of PEGylated lipids relative to the total amount of lipids in the lipid solution, on a molar basis, is at or between about 2% to about 20%. In various embodiments, the percentage of PEGylated lipids relative to the total amount of lipids is at or between 5 mole percent to about 15 mole percent.

In some embodiments, the lipids are 1, 2-dipalmitoyl-sn-glycero-3-phosphatidylcholine (DPPC), 1, 2-dipalmitoyl-sn-glycero-3-phosphatidic, mono sodium salt (DPPA), and N-(polyethylene glycol 5000 carbamoyl)-1, 2-dipalmitoyl-sn-glycero-3-phosphatidylethanolamine, monosodium salt (PEG5000-DPPE). PEG5000-DPPE may be MPEG5000-DPPE or HO-PEG5000-DPPE. In some embodiments, the lipids may be one, two or all three of DPPA, DPPC and PEG5000-DPPE. In some embodiments, the lipids may be one, two or all three of DPPC, DPPE and PEGylated-DPPE. In some embodiments, the lipids may be one, two or all three of DPPC, DPPE and PEG5000-DPPE. In these latter embodiments, the lipids may not include DPPA. PEG5000-DPPE may be MPEG5000-DPPE or HO-PEG5000-DPPE.

A wide variety of lipids, like those described in Unger et al. U.S. Pat. No. 5,469,854, may be used in this disclosure. Suitable lipids include, for example, fatty acids, lysolipids, fluorinated lipids, phosphocholines, such as those associated with platelet activation factors (PAF) (Avanti Polar Lipids, Alabaster, Ala.), including 1-alkyl-2-acetoyl-sn-glycero 3-phosphocholines, and 1-alkyl-2-hydroxy-sn-glycero 3-phosphocholines; phosphatidylcholine with both saturated and unsaturated lipids, including dioleoylphosphatidylcholine; dimyristoylphosphatidylcholine; dipentadecanoylphosphatidylcholine; dilauroylphosphatdylcholine; 1,2-dipalmitoyl-sn-glycero-3-phosphatidylcholine (DPPC); distearoylphosphatidylcholine (DSPC); and diarachidonylphosphatidylcholine (DAPC); phosphatidylethanolamines, such as dioleoylphosphatidylethanolamine, 1,2-dipalmitoyl-sn-glycero-3-phosphatidylethanolamine (DPPE) and distearoyl-phosphatidylethanolamine (DSPE); phosphatidylserine; phosphatidylglycerols, including distearoylphosphatidylglycerol (DSPG); phosphatidylinositol; sphingolipids such as sphingomyelin; glycolipids such as ganglioside GM1 and GM2; glucolipids; sulfatides; glycosphingolipids; phosphatidic acids, such as 1,2-dipalmitoyl-sn-glycero-3-phosphatidic acid (DPPA) and distearoylphosphatidic acid (DSPA); palmitic acid; stearic acid; arachidonic acid; and oleic acid.

Other suitable lipids include phosphatidylcholines, such as diolecylphosphatidylcholine, dimyristoylphosphatidylcholine, dipalmitoylphosphatidylcholine (DPPC), and distearoylphosphatidylcholine; phosphatidylethanolamines, such as dipalmitoylphosphatidylethanolamine (DPPE), dioleoylphosphatidylethanolamine and N-succinyl-dioleoylphosphatidylethanolamine; phosphatidylserines; phosphatidyl-glycerols; sphingolipids; glycolipids, such as ganglioside GM1; glucolipids; sulfatides; glycosphingolipids; phosphatidic acids, such as dipalmatoylphosphatidic acid (DPPA); palmitic fatty acids; stearic fatty acids; arachidonic fatty acids; lauric fatty acids; myristic fatty acids; lauroleic fatty acids; physeteric fatty acids; myristoleic fatty acids; palmitoleic fatty acids; petroselinic fatty acids; oleic fatty acids; isolauric fatty acids; isomyristic fatty acids; isopalmitic fatty acids; isostearic fatty acids; cholesterol and cholesterol derivatives, such as cholesterol hemisuccinate, cholesterol sulfate, and cholesteryl-(4′-trimethylammonio)-butanoate; polyoxyethylene fatty acid esters; polyoxyethylene fatty acid alcohols; polyoxyethylene fatty acid alcohol ethers; polyoxyethylated sorbitan fatty acid esters; glycerol polyethylene glycol oxystearate; glycerol polyethylene glycol ricinoleate; ethoxylated soybean sterols; ethoxylated castor oil; polyoxyethylene-polyoxypropylene fatty acid polymers; polyoxyethylene fatty acid stearates; 12-(((7′-diethylaminocoumarin-3-yl)-carbonyl)-methylamino)-octadecanoic acid; N-[12-(((7′-diethylamino-coumarin-3-yl)-carbonyl)-methyl-amino) octadecanoy 1]-2-amino-palmitic acid; 1,2-dioleoyl-sn-glycerol; 1,2-dipalmitoyl-sn-3-succinylglycerol; 1,3-dipalmitoyl-2-succinylglycerol; and 1-hexadecyl-2-palmitoyl-glycerophosphoethanolamine and palmitoylhomocysteine; lauryltrimethylammonium bromide (lauryl-=dodecyl-); cetyltrimethylammonium bromide (cetryl-=hexadecyl-); myristyltrimethylammonium bromide (myristyl-=tetradecyl-); alkyldimethylbenzylammonium chlorides, such as wherein alkyl is a C.sub.12, C.sub.14 or C.sub.16 alkyl; benzyldimethyldodecylammonium bromide; benzyldimethyldodecylammonium chloride, benzyldimethylhexadecylammonium bromide; benzyldimethylhexadecylammonium chloride; benzyldimethyltetradecylammonium bromide; benzyldimethyltetradecylammonium chloride; cetyldimethylethylammonium bromide; cetyldimethylethylammonium chloride; cetylpyridinium bromide; cetylpyridinium chloride; N-[1-2,3-dioleoyloxy)-propyl]-N,N,N-trimethylammonium chloride (DOTMA); 1,2-dioleoyloxy-3-(trimethylammonio) propane (DOTAP); and 1,2-diolcoyl-c-(4′-trimethylammonio)-butanoyl-sn-glycerol (DOTB).

In some embodiments where DPPA, DPPC and DPPE are used, their molar percentages may be about 77-90 mole % DPPC, about 5-15 mole % DPPA, and about 5-15 mole % DPPE, including DPPE-PEG5000. Preferred ratios of each lipid include those described in the Examples section such as a weight % ratio of 6.0 to 53.5 to 40.5 (DPPA:DPPC:MPEG5000-DPPE) or a mole % ratio of 10 to 82 to 8 (10:82:8) (DPPA:DPPC:MPEG5000-DPPE).

### Gas

The gas is preferably substantially insoluble in the lipid solutions provided herein. The gas may be a non-soluble fluorinated gas such as sulfur hexafluoride or a perfluorocarbon gas.

Examples of perfluorocarbon gases include perfluoropropane, perfluoromethane, perfluoroethane, perfluorobutane, perfluoropentane, perfluorohexane. Examples of gases that may be used in the microspheres of the invention are described in U.S. Pat. No. 5,656,211 and are incorporated by reference herein. In an important embodiment, the gas is perfluoropropane. Examples of gases include, but are not limited to, hexafluoroacetone, isopropylacetylene, allene, tetrafluoroallene, boron trifluoride, 1,2-butadiene, 1,3-butadiene, 1,2,3-trichlorobutadiene, 2-fluoro-1,3-butadiene, 2-methyl-1,3 butadiene, hexafluoro-1,3-butadiene, butadiyne, 1-fluorobutane, 2-methylbutane, decafluorobutane (perfluorobutane), decafluoroisobutane (perfluoroisobutane), 1-butene, 2-butene, 2-methy-1-butene, 3-methyl-1-butene, perfluoro-1-butene, perfluoro-1-butene, perfluoro-2-butene, 4-phenyl-3-butene-2-one, 2-methyl-1-butene-3-yne, butylnitrate, 1-butyne, 2-butyne, 2-chloro-1,1,1,4,4,4-hexafluoro-butyne, 3-methyl-1-butyne, perfluoro-2-butyne, 2-bromo-butyraldehyde, carbonyl sulfide, crotononitrile, cyclobutanc, methylcyclobutane, octafluorocyclobutanc (perfluorocyclobutanc), perfluoroisobutane, 3-chlorocyclopentene, cyclopropane, 1,2-dimethylcyclopropane, 1,1-dimethylcyclopropane, ethyl cyclopropane, methylcyclopropane, diacetylene, 3-ethyl-3-methyldiaziridine, 1,1,1-trifluorodiazoethane, dimethylamine, hexafluorodimethylamine, dimethylethylamine, bis-(dimethyl phosphine)amine, 2,3-dimethyl-2-norbornane, perfluoro-dimethylamine, dimethyloxonium chloride, 1,3-dioxolane-2-one, 1,1,1,1,2-tetrafluoroethane, 1,1,1-trifluoroethane, 1,1,2,2-tetrafluoroethane, 1,1,2-trichloro-1,2,2-trifluoroethane, 1,1-dichloroethane, 1,1-dichloro-1,2,2,2-tetrafluoroethane, 1,2-difluoroethane, 1-chloro-1,1,2,2,2-pentafluoroethane, 2-chloro-1,1-difluoroethane, 1-chloro-1,1,2,2-tetrafluoro-ethane, 2-chloro-1,1-difluorocthane, chlorocthane, chloropentafluoroethane, dichlorotrifluorocthane, fluoroethane, nitropentafluoroethane, nitrosopentafluoro-ethane, perfluoroethane, perfluoroethylamine, ethyl vinyl ether, 1,1-dichloroethylene, 1,1-dichloro-1,2-difluoro-ethylene, 1,2-difluoroethylene, methane, methane-sulfonyl-chlori-detrifluoro, methane-sulfonyl-fluoride-trifluoro, methane-(pentafluorothio)trifluoro, methane-bromo-difluoro-nitroso, methane-bromo-fluoro, methane-bromo-chloro-fluoro, methane-bromo-trifluoro, methane-chloro-difluoro-nitro, methane-chloro-dinitro, methane-chloro-fluoro, methane-chloro-trifluoro, methane-chloro-difluoro, methane-dibromo-difluoro, methane-dichloro-difluoro, methane-dichloro-fluoro, methane-difluoro, methane-difluoro-iodo, methane-disilano, methane-fluoro, methane-iodomethane-iodo-trifluoro, methane-nitro-trifluoro, methane-nitroso-triofluoro, methane-tetrafluoro, methane-trichloro-fluoro, methane-trifluoro, methanesulfenylchloride-trifluoro, 2-methyl butane, methyl ether, methyl isopropyl ether, methyl lactate, methyl nitrite, methyl sulfide, methyl vinyl ether, neopentane, nitrogen (N.sub.2), nitrous oxide, 1,2,3-nonadecane tricarboxylic acid-2-hydroxycrimethylester, 1-nonene-3-yne, oxygen (O.sub.2), oxygen 17 (.sup.17 O.sub.2), 1,4-pentadiene, n-pentane, dodecafluoropentane (perfluoropentane), tetradecafluorohexane (perfluorohexane), perfluoroisopentane, perfluoroncopentane, 2-pentanone-4-amino-4-methyl, 1-pentene, 2-pentene {cis}, 2-pentene {trans}, 1-pentene-3-bromo, 1-pentene-perfluoro, phthalic acid-tetrachloro, piperidine-2,3,6-trimethyl, propane, propane-1,1,1,2,2,3-hexafluoro, propane-1,2-epoxy, propane-2,2 difluoro, propane-2-amino, propane-2-chloro, propane-heptafluoro-1-nitro, propane-heptafluoro-1-nitroso, perfluoropropane, propene, propyl-1,1,1,2,3,3-hexafluoro-2,3 dichloro, propylene-1-chloro, propylene-chloro-{trans}, propylene-2-chloro, propylene-3-fluoro, propylene-perfluoro, propyne, propyne-3,3,3-trifluoro, styrene-3-fluoro, sulfur hexafluoride, sulfur (di)-decafluoro (S.sub.2 F.sub.10), tolueno-2,4-diamino, trifluoroacctonitrile, trifluoromethyl peroxide, trifluoromethyl sulfide, tungsten hexafluoride, vinyl acetylene, vinyl ether, neon, helium, krypton, xenon (especially rubidium enriched hyperpolarized xenon gas), carbon dioxide, helium, and air.

Fluorinated gases (that is, a gas containing one or more fluorine molecules, such as sulfur hexafluoride), fluorocarbon gases (that is, a fluorinated gas which is a fluorinated carbon or gas), and perfluorocarbon gases (that is, a fluorocarbon gas which is fully fluorinated, such as perfluoropropane and perfluorobutane) are preferred.

The gas such as the perfluorocarbon gas is typically present below its ordinary concentration at room temperature due to the incorporation of air during production. The concentration of perfluoropropane when present in a vial comprising a non-aqueous mixture and a gas headspace is expected to be about 6.52 mg/mL, at about one atmosphere of pressure. The concentrations of other gases, as known in the art, would be similarly diluted due to incorporation of air during production.

The gas, such as perflutren gas, may be injected into or otherwise added to the container (e.g., the vial) comprising the lipid formulation or into the lipid formulation itself in order to provide a gas other than air. Gases that are not heavier than air may be added to a sealed container while gases heavier than air may be added to a sealed or an unsealed container. It will be understood by one skilled in the art that a gaseous precursor may also be used, followed by conversion of the precursor into a gas either by temperature or pressure change.

### EXAMPLES

### Example 1

Vials of the aqueous lipid formulation (i.e., lipid suspension) DEFINITY® manufactured by Lantheus Medical Imaging, Inc., prior to activation, contained the following phospholipids: 1,2-dipalmitoyl-sn-glycero-3-phosphatidylcholine (DPPC; 0.401 mg/mL), 1,2-dipalmitoyl-sn-glycero-3-phosphatidic acid (DPPA; 0.045 mg/mL), and N-(methoxypolyethylene glycol 5000 carbamoyl)-1,2-dipalmitoyl-sn-glycero-3-phosphatidylethanolamine (MPEG5000 DPPE; 0.304 mg/ml) in a matrix of 103.5 mg/mL propylene glycol, 126.2 mg/mL glycerol (i.e., glycerin), and 2.34 mg/mL sodium phosphate monobasic monohydrate, 2.16 mg/mL sodium phosphate dibasic heptahydrate, and 4.87 mg/mL sodium chloride in Water for Injection. The pH is 6.2-6.8. The combined lipid concentration is 0.75 mg lipid per mL of formulation.

The volume of the lipid formulation was approximately 1.76 mL in a 2 cc (2 mL) Wheaton glass vial with an actual internal volume of about 3.8 mL and a head space of approximately 2.04 containing perfluoropropane gas (PFP, 6.52 mg/mL).

The commercially available, FDA-approved, aqueous based UCA formulation, DEFINITY® (Lantheus Medical Imaging, Inc.) is activated by mechanical shaking (described in U.S. Pat. No. 6,039,557, the content of which is hereby incorporated by reference and may be used in the present process) of the PFP/lipid formulation using a VIALMIX®. This results in incorporation of gas into lipid microspheres and represents the activated product (sce DEFINITY® Prescribing Information). Gas-filled bubbles can be analyzed for number and size distribution using a particle sizer (AccuSizer 780 or SpetraDyne) when diluted with salinc.

The acoustic attenuation assay was performed using a broadband transducer centered on frequencies of either 2.25 or 7.5 MHz.

### Example 2

Vials of non-aqueous lipid formulation (i.e., lipid solution) DEFINITY RT® manufactured by Lantheus Medical Imaging, Inc., prior to activation, contained the following phospholipids: 1,2-dipalmitoyl-sn-glycero-3-phosphatidylcholine (DPPC; 2.005 mg/mL), 1,2-dipalmitoyl-sn-glycero-3-phosphatidic acid (DPPA; 0.225 mg/mL), and N-(methoxypolyethylene glycol 5000 carbamoyl)-1,2-dipalmitoyl-sn-glycero-3-phosphatidylethanolamine (MPEG5000-DPPE; 1.520 mg/mL) in a matrix of 517.5 mg/mL propylene glycol, 631 mg/mL glycerol (i.e., glycerin), 0.370 mg/mL anhydrous sodium acetate, and 0.030 mg/mL glacial acetic acid. The pH is 5.2 to 6.4. The combined lipid concentration is 3.75 mg lipid per ml of formulation.

Prior to activation, the headspace of the vial contains 6.52 mg/mL octafluoropropane and the viscous solution contains 3.75 mg/mL of the lipid formulation.

The volume of the lipid formulation was approximately 0.35 mL in a 2 cc (2 mL) Wheaton glass vial with an actual internal volume of about 3.8 mL and a head space of approximately 3.45 mL containing perfluoropropane gas (PFP, 6.52 mg/mL).

Activation may be performed using VIALMIX® or VIALMIX-RFID®. This results in incorporation of gas into lipid nano/microbubbles which represent the activated product. Gas-filled bubbles can be analyzed for number and size distribution using a particle sizer (AccuSizer 780 or SpetraDyne) when diluted with saline.

The acoustic attenuation assay was performed using a broadband transducer centered on frequencies of either 2.25 or 7.5 MHz.

### Example 3

Vials of aqueous lipid formulation (e.g., DEFINITY®, Example 1) were shaken at 4530 rpm for differing time periods using a VIALMIX-RFID® modified to have programmable control of time and speed parameters. Samples were taken immediately after shaking and analyzed for gas bubble size distribution using an Accusizer 780. Bubbles in different size distributions indicate maximal formation of 0.5-1 μm microbubbles occurred with approximately 90 seconds of activation (Table 1), 1-2 μm at 45 seconds (data not shown) and 2-6 μm at 10 and 20 and 90 seconds (Table 3). Analysis of samples at each time point for acoustic attenuation demonstrated a maximal value for 7.5 MHz at 45 seconds, a profile that matches the 1-2 μm microbubble number. Similarly, the acoustic attenuation was near maximal for 2.25 MHz at 10 and 20 seconds, a profile that matched 2-6 μm microbubble number (data not shown).

### Example 4

Vials of aqueous lipid formulation (e.g., DEFINITY®, Example 1) were shaken at 6000 rpm for differing time periods using a VIALMIX-RFID® modified to have programmable control of time and speed parameters. Samples were taken immediately after shaking and analyzed for gas bubble size distribution using an Accusizer 780. Gas bubbles in different size distributions indicate maximal formation of 0.5-1 μm microbubbles occurred with approximately 20 and 25 seconds of activation (Table 2), 1-2 μm at 10-15 seconds (data not shown) and 2-6 μm at 5, 25 and 30 seconds. (Table 4). Analysis of samples at each time point for acoustic attenuation demonstrated a maximal value for 7.5 MHz at 10-15 seconds (data not shown), a profile that matches the 1-2 μm microbubble number. The acoustic attenuation was highest for 2.25 MHZ at 5 seconds and 25-30 seconds (data not shown), a profile that matches the 2-6 micron microbubble number.

### Example 5

Vials of non-aqueous lipid formulation (e.g., DEFINITY RT®, Example 2) were shaken at 4950 rpm for differing time periods using a VIALMIX-RFID® modified to have programmable control of time and speed parameters. Samples were taken immediately after shaking and analyzed for gas bubble size distribution using an Accusizer 780. Gas bubbles in different size distributions indicate maximal formation of 0.5-1 μm microbubbles occurred with approximately 250 seconds of activation (data not shown), 1-2 μm at 125 seconds (data not shown), and 2-6 μm at 80 seconds (data not shown). Analysis of samples at each time point for acoustic attenuation demonstrated a maximal value for 7.5 MHz at 100 to 150 seconds, a profile that matches the 1-2 μm microbubble number. Similarly the acoustic attenuation was maximal for 2.25 MHz at 80 seconds, a profile that matched 2-6 μm microbubble number (data not shown).

### Example 6

Vials of non-aqueous lipid formulation (e.g., DEFINITY RT®, Example 2) were shaken at 6000 rpm for differing time periods using a VIALMIX-RFID® modified to have programmable control of time and speed parameters. Samples were taken immediately after shaking and analyzed for gas bubble size distribution using an Accusizer 780. Gas bubbles in different size distributions indicate maximal formation of 0.5-1 μm microbubbles occurred with approximately 50-90 seconds of activation (Table 2A), 1-2 μm at 30-40 seconds (data not shown) and 2-6 μm at 20-90 seconds. (Table 4). Analysis of samples at each time point for acoustic attenuation demonstrated a maximal value for 7.5 MHz at 30 seconds (data not shown), a profile that matches the 1-2 μm microbubble number. Similarly the acoustic attenuation was maximal for 2.25 MHz at 20-90 seconds (Table 2A), a profile that matched 2-6 μm microbubble number.

Vials of non-aqueous lipid formulation (e.g., DEFINITY RT®, Example 2) were shaken at speeds between 6200 and 6600 rpm for differing time periods using a VIALMIX-RFID® modified to have programmable control of time and speed parameters using a Trinamic brushless motor. Samples were taken immediately after shaking, inverted 10 times, and analyzed for gas bubble size distribution using an Accusizer 780. Gas bubbles in different size distributions indicate maximal formation of 0.5-1 μm microbubbles occurred with approximately 30-60 seconds of activation with speeds between 6200 to 6600 rpm (Table 2B), 1-2 μm around 15 seconds (data not shown) and 2-6 μm at 15 to 70 seconds (data not shown).

The data show that non-aqueous lipid formulations (e.g., DEFINITY RT®, Example 2) can be sufficiently activated at 6000 rpm, at 6200 rpm, and at 6600 rpm, albeit at different peak times. At 6000 rpm and 6200 rpm, peak times appear to be about 60 seconds. At 6600 rpm, maximum effect is seen at about 15 to about 30 seconds.

### Example 7

Vials of non-aqueous lipid formulation (e.g., DEFINITY RT®, Example 2) were either a) un-modified or b) contained 1.5 times the fill volume of the non-aqueous lipid formulation (i.e., 1.5 times 0.35 mL lipid formulation) (referred to as DEFINITY-RT 1.5× fill volume, Table 5) or c) was diluted by adding 0.5 times the fill volume with propylene glycol/glycerol (50:50) matrix alone (i.e., 0.35 mL lipid formulation and about 0.18 mL matrix) (referred to as DEFINITY-RT 1×+0.5*, Table 5). Higher numbers of 2-6 μm microbubbles could be formed by increasing the amount of non-aqueous lipid solution or surprisingly adding only matrix. These changes increased the acoustic attenuation at 2.25 MHz but not at 7.5 MHz (Table 5). Analysis of the size distribution for gas bubbles using the Accusizer 780 indicated the majority of gas bubbles were less than 2 μm when shaken at 6000 rpm for 60 seconds (data not shown). However with a dilution of the matrix, the distribution contained substantially more microbubbles above 2 μm but less than 10 μm. This distribution produced a pronounced increase in ultrasound attenuation at 2.25 MHz (Table 5).

### Example 8

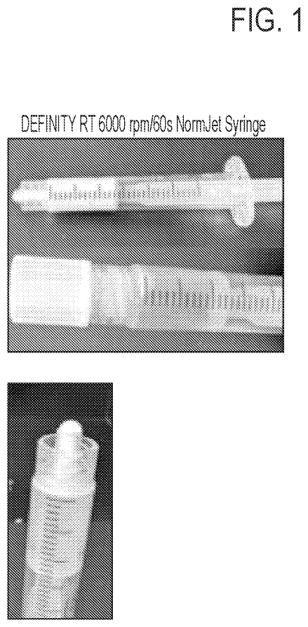

Vials of non-aqueous lipid formulation (e.g., DEFINITY RT®, Example 2) were shaken at 6000 rpm for 60 seconds using a VIALMIX-RFID®. After dilution with saline, samples were inverted ten times and either (a) taken immediately and analyzed for gas bubble size distribution using an Accusizer 780 or (b) transferred to a 3 mL (or a 5 mL syringe) Becton Dickinson or Norm Jet syringe, with a leur-lok plug inserted, and plunger pressure applied by hand until the white milky solution became clear (see FIG. 1 for 3 mL syringe). After ten inversions, the plug was removed and a sample analyzed for gas bubble size distribution using an Accusizer 780. Sufficient hand pressure and release resulted in a marked clearance of bubbles greater than 500 nm (see Table 6).

Vials of aqueous lipid formulation (e.g., DEFINITY®, Example 1) were shaken at 4530 rpm for 45 seconds using a VIALMIX-RFID®. Samples were either (a) taken immediately after shaking and inversion, and analyzed for gas bubble size distribution using an Accusizer 780 or (b) transferred to a 3 mL Becton Dickinson or Norm Jet syringe, with a leur-lok plug inserted, and plunger pressure applied by hand until the white milky solution became clear. After ten inversions, the plug was removed and a sample was analyzed for gas bubble size distribution using an Accusizer 780. Sufficient hand pressure and release resulted in a marked clearance of bubbles greater than 500 nm (see Table 6).

Some studies examined the use of a device to aid in pressurizing the syringe after activation. A Caulk gun was adapted to allow insertion of a medical syringe and to facilitate and aid in controlling the pressurization. Additional pressurizing devices involving mechanically controlled plunger using gears and/or a step motor with a measured pressure feedback is contemplated.

TABLE 1

| | Shake | | Total | |
|---|---|---|---|---|
| Lipid | speed | Shake time | bubble Conc. | % Nano- |
| Formulation | (RPM) | (seconds) | (0.5-500 μm) | bubbles |
| DEFINITY RT | 4950 | 10 | 4.24E+08 | 56.4 |
| DEFINITY RT | 4950 | 20 | 1.84E+09 | 54.0 |
| DEFINITY RT | 4950 | 30 | 3.44E+09 | 50.8 |
| DEFINITY RT | 4950 | 45 | 4.44E+09 | 51.1 |
| DEFINITY RT | 4950 | 45 | 4.91E+09 | 50.8 |
| DEFINITY RT | 4950 | 45 | 7.03E+09 | 50.8 |
| DEFINITY RT | 4950 | 60 | 9.37E+09 | 50.2 |
| DEFINITY RT | 4950 | 75 | 1.26E+10 | 49.3 |
| DEFINITY RT | 4950 | 90 | 1.55E+10 | 49.7 |
| DEFINITY RT | 4950 | 120 | 1.98E+10 | 53.8 |
| DEFINITY | 4530 | 10 | 2.89E+09 | 61.0 |
| DEFINITY | 4530 | 20 | 7.95E+09 | 61.3 |
| DEFINITY | 4530 | 30 | 1.14E+10 | 63.4 |
| DEFINITY | 4530 | 45 | 1.50E+10 | 65.3 |
| DEFINITY | 4530 | 45 | 1.47E+10 | 66.4 |
| DEFINITY | 4530 | 45 | 1.78E+10 | 66.2 |
| DEFINITY | 4530 | 60 | 1.84E+10 | 71.4 |
| DEFINITY | 4530 | 75 | 1.67E+10 | 71.0 |
| DEFINITY | 4530 | 90 | 1.16E+10 | 83.0 |

TABLE 2A

| | Shake | | Total | |
|---|---|---|---|---|
| Lipid | speed | Shake time | bubble Conc. | % Nano- |
| Formulation | (RPM) | (seconds) | (0.5-500 μm) | bubbles |
| DEFINITY RT | 4950 | 45 | 4.90E+09 | 49.87 |
| DEFINITY RT | 6000 | 10 | 1.97E+09 | 56.14 |
| DEFINITY RT | 6000 | 15 | 5.28E+09 | 54.52 |
| DEFINITY RT | 6000 | 20 | 7.88E+09 | 56.23 |
| DEFINITY RT | 6000 | 30 | 1.72E+10 | 59.84 |
| DEFINITY RT | 6000 | 40 | 2.37E+10 | 68.33 |
| DEFINITY RT | 6000 | 50 | 2.59E+10 | 77.92 |
| DEFINITY RT | 6000 | 60 | 2.62E+10 | 84.55 |
| DEFINITY RT | 6000 | 60 | 2.09E+10 | 88.99 |
| DEFINITY RT | 6000 | 60 | 2.75E+10 | 87.57 |
| DEFINITY RT | 6000 | 75 | 1.99E+10 | 93.39 |
| DEFINITY RT | 6000 | 90 | 1.46E+10 | 93.68 |
| DEFINITY | 4530 | 45 | 1.19E+10 | 65.40 |
| DEFINITY | 6000 | 5 | 5.34E+09 | 59.16 |
| DEFINITY | 6000 | 10 | 1.01E+10 | 62.51 |
| DEFINITY | 6000 | 15 | 1.30E+10 | 67.37 |
| DEFINITY | 6000 | 20 | 1.47E+10 | 80.69 |
| DEFINITY | 6000 | 20 | 1.30E+10 | 76.83 |
| DEFINITY | 6000 | 20 | 1.32E+10 | 78.34 |
| DEFINITY | 6000 | 25 | 1.22E+10 | 84.53 |
| DEFINITY | 6000 | 30 | 3.77E+09 | 71.41 |

TABLE 2B

| | Shake | | Total | |
|---|---|---|---|---|
| | speed | Shake time | bubble Conc. | % Nano- |
| UCA | (RPM) | (seconds) | (0.5-500 μm) | bubbles |
| DEFINITY RT | 4950 | 45 | 5.00E+09 | 54.9 |
| DEFINITY RT | 6000 | 60 | 2.26E+10 | 80.6 |
| DEFINITY RT | 6200 | 60 | 1.72E+10 | 97.0 |
| DEFINITY RT | 6600 | 15 | 1.19E+10 | 70.3 |
| DEFINITY RT | 6600 | 30 | 1.41E+10 | 96.3 |
| DEFINITY RT | 6600 | 45 | 1.27E+9 | 54% |
| DEFINITY RT | 6600 | 70 | 1.09E+9 | 38.5 |

TABLE 3

| | | | Total | |
|---|---|---|---|---|
| | Shake | | microbubbles | % 2-6 um |
| Lipid | speed | Shake time | Conc. | micro- |
| Formulation | (RPM) | (seconds) | (1-500 μm) | bubbles |
| DEFINITY RT | 4950 | 10 | 1.81E+08 | 12.6 |
| DEFINITY RT | 4950 | 20 | 8.31E+08 | 13.2 |
| DEFINITY RT | 4950 | 30 | 1.66E+09 | 14.6 |
| DEFINITY RT | 4950 | 45 | 2.14E+09 | 11.6 |
| DEFINITY RT | 4950 | 45 | 2.38E+09 | 12.7 |
| DEFINITY RT | 4950 | 45 | 3.40E+09 | 12.3 |
| DEFINITY RT | 4950 | 60 | 4.59E+09 | 10.8 |
| DEFINITY RT | 4950 | 75 | 6.30E+09 | 7.1 |
| DEFINITY RT | 4950 | 90 | 7.68E+09 | 4.4 |
| DEFINITY RT | 4950 | 120 | 8.91E+09 | 2.3 |
| DEFINITY | 4530 | 10 | 1.10E+09 | 13.7 |
| DEFINITY | 4530 | 20 | 3.01E+09 | 12.4 |
| DEFINITY | 4530 | 30 | 4.08E+09 | 7.2 |
| DEFINITY | 4530 | 45 | 5.08E+09 | 4.3 |
| DEFINITY | 4530 | 45 | 4.81E+09 | 3.4 |
| DEFINITY | 4530 | 45 | 5.85E+09 | 3.9 |
| DEFINITY | 4530 | 60 | 5.08E+09 | 3.7 |
| DEFINITY | 4530 | 75 | 4.70E+09 | 3.4 |
| DEFINITY | 4530 | 90 | 1.90E+09 | 10.6 |

TABLE 4

| | | | Total | |
|---|---|---|---|---|
| | Shake | | microbubbles | % 2-6 um |
| Lipid | speed | Shake time | Conc. | micro- |
| Formulation | (RPM) | (seconds) | (1-500 μm) | bubbles |
| DEFINITY RT | 4950 | 45 | 2.42E+09 | 13.4 |
| DEFINITY RT | 6000 | 10 | 8.46E+08 | 5.5 |
| DEFINITY RT | 6000 | 15 | 2.35E+09 | 4.5 |
| DEFINITY RT | 6000 | 20 | 3.37E+09 | 4.1 |
| DEFINITY RT | 6000 | 30 | 6.73E+09 | 2.9 |
| DEFINITY RT | 6000 | 40 | 7.21E+09 | 2.4 |
| DEFINITY RT | 6000 | 50 | 5.42E+09 | 2.5 |
| DEFINITY RT | 6000 | 60 | 3.78E+09 | 4.0 |
| DEFINITY RT | 6000 | 60 | 2.13E+09 | 8.5 |
| DEFINITY RT | 6000 | 60 | 3.16E+09 | 5.3 |
| DEFINITY RT | 6000 | 75 | 1.23E+09 | 19.1 |
| DEFINITY RT | 6000 | 90 | 8.89E+08 | 27.5 |
| DEFINITY | 4530 | 45 | 4.00E+09 | 4.0 |
| DEFINITY | 6000 | 5 | 2.14E+09 | 12.6 |
| DEFINITY | 6000 | 10 | 3.69E+09 | 5.8 |
| DEFINITY | 6000 | 15 | 4.13E+09 | 5.0 |
| DEFINITY | 6000 | 20 | 2.72E+09 | 7.7 |
| DEFINITY | 6000 | 20 | 2.90E+09 | 6.6 |
| DEFINITY | 6000 | 20 | 2.75E+09 | 6.5 |
| DEFINITY | 6000 | 25 | 1.82E+09 | 10.7 |
| DEFINITY | 6000 | 30 | 1.06E+09 | 18.5 |

TABLE 5

| | | | | Total | | | | | 2.25 | 7.5 |
|---|---|---|---|---|---|---|---|---|---|---|
| | Shake | Shake | | Conc. | | | % micro- | % micro- | MHz | MHz |
| Lipid | Speed | Time | Fill | (1-500 | 2 ≤ | 6 ≤ | bubble | bubble | atten- | Atten- |
| Formulation | (RPM) | (Sec) | volume | um) | P < 6 | P < 10 | 2-6 | 6-10 | uation | uation |
| DEFINITY RT | 4950 | 45 | 1X | 2.8E+09 | 3.6E+08 | 3.5E+07 | 12.8 | 1.2 | 0.50 | 0.49 |
| DEFINITY RT | 4950 | 45 | 1X | 2.5E+09 | 2.7E+08 | 1.1E+07 | 10.7 | 0.4 | 0.54 | 0.58 |
| DEFINITY RT | 4950 | 45 | 1X | 2.7E+09 | 3.2E+08 | 2.0E+07 | 11.8 | 0.8 | 0.52 | 0.54 |
| DEFINITY RT | 4950 | 45 | 1X | 2.2E+09 | 2.7E+08 | 2.4E+07 | 12.4 | 1.1 | 0.56 | 0.57 |
| DEFINITY RT | 4950 | 45 | 1X | 2.7E+09 | 2.8E+08 | 2.6E+07 | 10.5 | 1.0 | 0.51 | 0.55 |
| DEFINITY RT | 4950 | 45 | 1X | 2.5E+09 | 2.4E+08 | 1.9E+07 | 9.6 | 0.8 | 0.57 | 0.59 |
| DEFINITY RT | 4950 | 45 | 1X | 2.2E+09 | 2.7E+08 | 1.9E+07 | 12.4 | 0.9 | 0.66 | 0.59 |
| DEFINITY RT | 4950 | 45 | 1X | 2.5E+09 | 2.9E+08 | 1.7E+07 | 11.8 | 0.7 | 0.42 | 0.42 |
| DEFINITY RT | 4950 | 45 | 1X | 2.5E+09 | 3.1E+08 | 1.5E+07 | 12.5 | 0.6 | 0.50 | 0.48 |
| DEFINITY RT | 4950 | 45 | 1.5X | 1.3E+10 | 4.1E+08 | 4.2E+07 | 3.2 | 0.3 | 0.84 | 1.04 |
| DEFINITY RT | 4950 | 45 | 1.5X | 4.1E+09 | 3.9E+08 | 2.7E+07 | 9.4 | 0.7 | 0.87 | 1.12 |
| DEFINITY RT | 6000 | 60 | 1X | 1.1E+09 | 7.0E+07 | 3.4E+07 | 6.3 | 3.1 | 0.32 | 0.10 |
| DEFINITY RT | 6000 | 60 | 1X | 3.9E+09 | 1.0E+08 | 1.3E+07 | 2.7 | 0.3 | 0.33 | 0.23 |
| DEFINITY RT | 6000 | 60 | 1.5X | 1.0E+09 | 3.7E+08 | 9.6E+07 | 36.7 | 9.6 | >1.24 | 0.25 |
| DEFINITY RT | 6000 | 60 | 1.5X | 1.2E+09 | 3.9E+08 | 8.8E+07 | 31.7 | 7.1 | >1.24 | 0.40 |
| DEFINITY RT | 6000 | 60 | 1.5X | 1.2E+09 | 3.2E+08 | 7.2E+07 | 27.1 | 6.1 | >1.24 | 0.38 |
| DEFINITY RT | 6000 | 60 | 1.5X | 1.0E+09 | 4.4E+08 | 1.0E+08 | 42.8 | 10.1 | >1.24 | 0.23 |
| DEFINITY RT | 6000 | 60 | 1.5X | 1.8E+09 | 9.4E+08 | 9.5E+07 | 51.7 | 5.2 | >1.24 | 0.32 |
| DEFINITY RT | 6000 | 60 | 1.5X | 1.4E+09 | 6.6E+08 | 6.3E+07 | 47.2 | 4.5 | >1.24 | 0.30 |
| DEFINITY RT | 6000 | 60 | 1.5X | 1.3E+09 | 5.7E+08 | 1.4E+08 | 42.9 | 10.2 | 1.46 | 0.33 |
| DEFINITY RT | 6000 | 60 | 1.5X | 1.3E+09 | 6.5E+08 | 1.6E+08 | 50.4 | 12.2 | 1.19 | 0.30 |
| DEFINITY RT | 6000 | 60 | 1.5X | 1.5E+09 | 6.7E+08 | 1.1E+08 | 46.1 | 7.7 | 1.22 | 0.34 |
| DEFINITY RT | 6000 | 60 | 1.5X | 1.8E+09 | 8.4E+08 | 1.1E+08 | 46.6 | 6.2 | 1.31 | 0.38 |
| DEFINITY RT | 6000 | 60 | 1.5X | 2.0E+09 | 9.4E+08 | 8.5E+07 | 46.3 | 4.2 | 1.68 | 0.39 |
| DEFINITY RT | 6000 | 60 | 1.5X | 1.4E+09 | 7.8E+08 | 1.2E+08 | 55.0 | 8.7 | 1.58 | 0.36 |
| DEFINITY RT | 6000 | 60 | 1X + 0.5* | 9.7E+08 | 3.8E+08 | 8.5E+07 | 39.5 | 8.8 | >1.24 | 0.22 |
| DEFINITY RT | 6000 | 60 | 1X + 0.5* | 9.5E+08 | 3.5E+08 | 7.5E+07 | 36.9 | 7.9 | >1.24 | 0.29 |
| DEFINITY RT | 6000 | 60 | 1X + 0.5* | 7.0E+08 | 3.0E+08 | 7.4E+07 | 42.6 | 10.5 | 1.12 | 0.16 |
| DEFINITY RT | 6000 | 60 | 1X + 0.5* | 1.1E+09 | 5.3E+08 | 8.9E+07 | 50.4 | 8.4 | >1.24 | 0.23 |
| DEFINITY RT | 6000 | 60 | 1X + 0.5* | 1.1E+09 | 5.7E+08 | 8.5E+07 | 51.5 | 7.6 | >1.24 | 0.26 |
| DEFINITY RT | 6000 | 60 | 1X + 0.5* | 1.4E+09 | 6.7E+08 | 7.6E+07 | 47.4 | 5.4 | >1.24 | 0.25 |
| DEFINITY RT | 6000 | 60 | 1X + 0.5* | 1.2E+09 | 6.7E+08 | 9.6E+07 | 53.9 | 7.7 | 1.31 | 0.27 |
| DEFINITY RT | 6000 | 60 | 1X + 0.5* | 1.2E+09 | 6.7E+08 | 9.5E+07 | 55.9 | 7.9 | 1.25 | 0.27 |
| DEFINITY RT | 6000 | 60 | 1X + 0.5* | 1.3E+09 | 6.9E+08 | 9.0E+07 | 55.0 | 7.2 | 1.09 | 0.28 |

*(50/50 PG/G)

TABLE 6

| | | | Total | |
|---|---|---|---|---|
| | Shake | Shake | bubble | |
| | speed | time | Conc. | |
| UCA | (RPM) | (seconds) | (0.5-500 μm) | Treatment |
| DEFINITY | 4530 | 45 | 1.38E+10 | None pressurized and |
| | | | | inverted 10 times |
| DEFINITY | 4530 | 45 | 1.70E+08 | Pressurized, released |
| | | | | and inverted 10 times |
| DEFINITY | 4530 | 45 | 1.81E+08 | Pressurized, released |
| | | | | and inverted 10 times |
| DEFINITY | 6000 | 60 | 1.34E+10 | None pressurized and |
| RT | | | | inverted 10 times |
| DEFINITY | 6000 | 60 | 2.47E+10 | None pressurized and |
| RT | | | | inverted 10 times |
| DEFINITY | 6000 | 60 | 1.43E+10 | None pressurized and |
| RT | | | | inverted 10 times |
| DEFINITY | 6000 | 60 | 6.96 + 07 | Pressurized, released |
| RT | | | | and inverted 10 times |
| DEFINITY | 6000 | 60 | 5.22E+7 | Pressurized, released |
| RT | | | | and inverted 10 times |

### Clauses and Embodiments

This disclosure therefore provides a number of inventive embodiments, as provided below in clause form;
Clause 1. A composition comprising
   a population of lipid-encapsulated perfluorocarbon gas bubbles, wherein 75% or more of the lipid-encapsulated perfluorocarbon gas bubbles are nanobubbles having a diameter in the range of about 100 nanometers to less than or about 1 micron, and wherein the lipid-encapsulated perfluorocarbon gas bubbles comprise DPPC and PEG-DPPE, optionally wherein the nanobubbles are present in a concentration range of about 7.0×10⁹ to about 2.5×10¹⁰ nanobubbles per mL, or about 1×10¹⁰ to about 2.5×10¹⁰ nanobubbles per mL.
Clause 2. The composition of clause 1, wherein 80% or more of the lipid-encapsulated perfluorocarbon gas bubbles are nanobubbles having a diameter in the range of about 100 nanometers to less than or about 1 micron.
Clause 3. The composition of clause 1 or 2, wherein the lipid-encapsulated perfluorocarbon gas bubbles comprise DPPA, DPPC and PEG-DPPE, optionally in a mole % ratio of about 10 to about 82 to about 8 including 10:82:8, or wherein the lipid formulation comprises DPPE, DPPC and PEG-DPPE, optionally in a mole % ratio of about 10 to about 82 to about 8 including 10:82:8.
Clause 4, The composition of any one of clauses 1-3, wherein PEG-DPPE is PEG5000-DPPE.
Clause 5. The composition of any one of clauses 1-4, wherein PEG-DPPE is MPEG5000-DPPE.
Clause 6. The composition of any one of clauses 1-5, wherein the lipid-encapsulated perfluorocarbon gas bubbles are lipid-encapsulated perfluoropropane gas bubbles.
Clause 7. The composition of any one of clauses 1-6, wherein the population of lipid-encapsulated perfluorocarbon gas bubbles is present in a non-aqueous lipid solution comprising DPPA, DPPC and PEG-DPPE, glycerol, propylene glycol, and less than or equal to 5% water (v/v), or wherein the population of lipid-encapsulated perfluorocarbon gas bubbles is present in a non-aqueous lipid solution comprising DPPE, DPPC and PEG-DPPE, glycerol, propylene glycol, and less than or equal to 5% water (v/v).
Clause 8. The composition of clause 7, wherein the lipids are present in a combined concentration of about 3.75 mg/ml.
Clause 9. The composition of clause 7, wherein the lipids are present in a combined concentration of about 2.5 mg/ml.
Clause 10. The composition of any one of clauses 1-6, wherein the population of lipid-encapsulated perfluorocarbon gas bubbles is present in an aqueous lipid suspension comprising glycerol and propylene glycol.
Clause 11. The composition of clause 10, wherein the lipid suspension has a combined lipid concentration of about 0.75 mg/ml.
Clause 12. The composition of any one of clauses 1-11, wherein the population of lipid-encapsulated perfluorocarbon gas bubbles lacks DPPA.
Clause 13. A method for forming lipid-encapsulated perfluorocarbon gas bubbles comprising
   activating a lipid formulation comprising DPPC and PEG-DPPE, glycerol and propylene glycol, in the presence of a perfluorocarbon gas, at a shaking rate and shaking time sufficient to yield a population of lipid-encapsulated perfluorocarbon gas bubbles wherein 75% or more of the gas bubbles are nanobubbles having a diameter in the range of about 100 nanometers to about 1 micron.
Clause 14. The method of clause 13, wherein 80% or more of the lipid-encapsulated perfluorocarbon gas bubbles are nanobubbles having a diameter in the range of about 100 nanometers to about 1 micron.
Clause 15. The method of clause 13 or 14, wherein the lipid formulation comprises DPPA, DPPC and PEG-DPPE, optionally in a mole % ratio of about 10 to about 82 to about 8 including 10:82:8, or wherein the lipid formulation comprises DPPE, DPPC and PEG-DPPE, optionally in a mole % ratio of about 10 to about 82 to about 8 including 10:82:8.
Clause 16. The method of any one of clauses 13-15, wherein PEG-DPPE is PEG5000-DPPE,
Clause 17. The method of any one of clauses 13-15, wherein PEG-DPPE is MPEG5000-DPPE.
Clause 18. The method of any one of clauses 13-17, wherein the perfluorocarbon gas is perfluoropropane gas, and the lipid-encapsulated perfluorocarbon gas bubbles are lipid-encapsulated perfluoropropane gas bubbles.
Clause 19. The method of any one of clauses 13-18, wherein the lipid formulation is a non-aqueous lipid solution comprising DPPA, DPPC and PEG-DPPE, glycerol, propylene glycol, and less than or equal to 5% water (v/v), or wherein the lipid formulation is a non-aqueous lipid solution comprising DPPE, DPPC and PEG-DPPE, glycerol, propylene glycol, and less than or equal to 5% water (v/v),
Clause 20. The method of clause 19, wherein the lipid formulation has a combined lipid concentration of about 3.75 mg/ml.
Clause 21. The method of clause 19 or 20, wherein the lipid formulation is activated at a shaking rate of about 6000 rpm for a shaking time of about 50 seconds to about 90 seconds.
Clause 22. The method of clause 19 or 20, wherein the lipid formulation is activated at a shaking rate of about 6000 rpm for a shaking time of about 50 seconds, about 60 seconds, about 75 seconds, or about 90 seconds.
Clause 23. The method of clause 19, wherein the lipid formulation has a combined lipid concentration of about 2.5 mg/ml.
Clause 24. The method of clause 19, wherein the lipid formulation is present in a Wheaton 2 mL glass vial having an internal volume of about 3.8 ml, and comprising about 0.35 ml of lipid formulation, and about 3.45 ml of gas, or wherein the lipid formulation is present in a Wheaton 2 mL glass vial having an internal volume of about 3.8 ml, and comprising about 0.52 ml of lipid formulation, and about 3.28 ml of gas.
Clause 25. The method of clause 19, wherein the lipid formulation is present in a Wheaton 2 mL glass vial having an internal volume of about 3.8 ml, wherein about 9% of the internal volume is lipid occupied by lipid formulation prior to activation, or wherein about 14% of the internal volume is occupied by lipid formulation prior to activation.
Clause 26. The method of any one of clauses 13-18, wherein the lipid formulation is an aqueous lipid suspension comprising DPPA, DPPC and PEG-DPPE, glycerol, and propylene glycol, or wherein the lipid formulation is an aqueous lipid suspension comprising DPPE, DPPC and PEG-DPPE, glycerol, and propylene glycol.
Clause 27. The method of clause 26, wherein the lipid formulation has a combined lipid concentration of about 0.75 mg/ml.
Clause 28. The method of clause 26 or 27, wherein the lipid formulation is activated in the presence of gas at a shaking rate of about 4530 rpm for a shaking time of about 90 seconds.
Clause 29. The method of clause 26 or 27, wherein the lipid formulation is activated in the presence of gas at a shaking rate of about 6000 rpm for a shaking time of about 20 seconds to about 25 seconds.
Clause 30. The method of clause 26 or 27, wherein the lipid formulation is activated in the presence of gas at a shaking rate of about 6000 rpm for a shaking time of about 20 seconds or about 25 seconds.
Clause 31. The method of any one of clauses 13-30, wherein the lipid formulation lacks DPPA.
Clause 32. A population of lipid-encapsulated perfluorocarbon gas bubbles formed according to the method of any one of clauses 13-31.
Clause 33. A method of treating a subject having a condition, comprising
   administering to a subject having a condition an effective amount of a population of lipid-encapsulated perfluorocarbon gas bubbles, wherein 75% or more of the bubbles in the population are nanobubbles having a diameter in the range of about 100 nanometers to about 1 micron, and wherein the lipid-encapsulated perfluorocarbon gas bubbles comprise DPPC and PEG-DPPE, and
   applying ultrasound energy to a region of the subject affected by the condition.
Clause 34. In a method of improving tissue perfusion in a subject comprising exposing the subject to therapeutic ultrasound, the improvement comprising
   administering to the subject a population of lipid-encapsulated perfluorocarbon gas bubbles wherein 75% or more of the bubbles in the population are nanobubbles having a diameter in the range of about 100 nanometers to about 1 micron before and/or during exposure to the therapeutic ultrasound.
Clause 35. The method of clause 33, wherein the condition is presence of a thrombus/blood clot, vascular occlusion, retinal vein occlusion, a tumor, erectile dysfunction, Alzheimer's disease, Parkinson's disease.
Clause 36. The method of clause 33, wherein the condition is one that benefits from disruption of the blood-brain-barrier (BBB), optionally wherein a condition-specific therapeutic agent is also administered to the subject, before, during and/or after the population of gas bubbles and/or before, during and/or after exposure to the therapeutic ultrasound.
Clause 37. The method of any one of clauses 33-36, wherein the lipid-encapsulated perfluorocarbon gas nanobubbles experience stable cavitation in vivo after application of the ultrasound energy.
Clause 38. The method of any one of clauses 33-36, wherein the lipid-encapsulated perfluorocarbon gas nanobubbles experience inertial cavitation in vivo after application of the ultrasound energy, optionally as detected and/or measured by emitted broadband radiofrequency.
Clause 39. The method of any one of clauses 33-38, wherein 80% or more of the lipid-encapsulated perfluorocarbon gas bubbles are nanobubbles having a diameter in the range of about 100 nanometers to about 1 micron.
Clause 40. The method of any one of clauses 33-39, wherein the lipid-encapsulated perfluorocarbon gas bubbles comprise DPPA, DPPC and PEG-DPPE, optionally in a mole % ratio of about 10 to about 82 to about 8 including 10:82:8, or wherein the lipid formulation comprises DPPE, DPPC and PEG-DPPE, optionally in a mole % ratio of about 10 to about 82 to about 8 including 10:82:8.
Clause 41. The method of any one of clauses 33-40, wherein PEG-DPPE is PEG5000-DPPE.
Clause 42. The method of any one of clauscs 33-40, wherein PEG-DPPE is MPEG5000-DPPE.
Clause 43. The method of any one of clauses 33-42, wherein the lipid-encapsulated perfluorocarbon gas bubbles are lipid-encapsulated perfluoropropane gas bubbles.
Clause 44. The method of any one of clauses 33-43, wherein the population of lipid-encapsulated perfluorocarbon gas bubbles is formed according to the method of any one of clauses 13-31.
Clause 45. The method of any one of clauses 33-43, wherein the population of lipid-encapsulated perfluorocarbon gas bubbles is a composition of any one of clauses 1-12.
Clause 46. A composition comprising
   a population of lipid-encapsulated perfluorocarbon gas bubbles, wherein 15% or more of microbubbles in the population have a diameter of about 2 microns to less than or about 6 microns, and
   wherein the lipid-encapsulated perfluorocarbon gas bubbles comprise DPPC and PEG-DPPE, optionally wherein the microbubbles having a diameter of about 2 microns to less than or about 6 microns are present in a concentration range of about 0.24×10⁹ to about 2.44×10⁹ bubbles per mL.
Clause 47. The composition of clause 46, wherein 20% or more of the lipid-encapsulated perfluorocarbon gas microbubbles in the population have a diameter of about 2 microns to less than or about 6 microns.
Clause 48. The composition of clause 46 or 47, wherein the lipid-encapsulated perfluorocarbon gas bubbles comprise DPPA, DPPC and PEG-DPPE, optionally in a mole % ratio of about 10 to about 82 to about 8 including 10:82:8, or wherein the lipid-encapsulated perfluorocarbon gas bubbles comprise DPPE, DPPC and PEG-DPPE, optionally in a mole % ratio of about 10 to about 82 to about 8 including 10:82:8.
Clause 49. The composition of any one of clauses 46-48, wherein PEG-DPPE is PEG5000-DPPE.
Clause 50. The composition of any one of clauses 46-49, wherein PEG-DPPE is MPEG5000-DPPE.
Clause 51. The composition of any one of clauses 46-50, wherein lipid-encapsulated perfluorocarbon gas bubbles are lipid-encapsulated perfluoropropane gas bubbles.
Clause 52. The composition of any one of clauses 46-51, wherein the population of lipid-encapsulated perfluorocarbon gas bubbles is present in a non-aqueous lipid solution comprising DPPA, DPPC and PEG-DPPE, glycerol, propylene glycol, and less than or equal to 5% water (v/v), or wherein the population of lipid-encapsulated perfluorocarbon gas bubbles is present in a non-aqueous lipid solution comprising DPPE, DPPC and PEG-DPPE, glycerol, propylene glycol, and less than or equal to 5% water (v/v).
Clause 53. The composition of any one of clauses 46-52, wherein the lipids are present in a combined concentration of about 3.75 mg/ml.
Clause 54. The composition of any one of clauses 46-52, wherein the lipids are present in a combined concentration of about 2.5 mg/ml.
Clause 55. The composition of any one of clauses 46-51, wherein the population of lipid-encapsulated perfluorocarbon gas bubbles is present in an aqueous lipid suspension comprising glycerol and propylene glycol.
Clause 56. The composition of clause 55, wherein the lipid suspension has a combined lipid concentration of about 0.75 mg/ml.
Clause 57. The composition of any one of clauses 46-56, wherein the population of lipid-encapsulated perfluorocarbon gas bubbles exhibits an acoustic attenuation, of 2.25 MHz ultrasound frequency, in the range of greater than about 1, optionally greater than about 1.2, optionally in the range of about 1 to about 1.7.
Clause 58. The composition of any one of clauses 46-57, wherein the population of lipid-encapsulated perfluorocarbon gas bubbles lacks DPPA.
Clause 59. A method for forming lipid-encapsulated perfluorocarbon gas bubbles comprising
   activating a lipid formulation comprising DPPC and PEG-DPPE, glycerol and propylene glycol, in the presence of a perfluorocarbon gas, at a shaking rate and a shaking time sufficient to yield a population of lipid-encapsulated perfluorocarbon gas microbubbles wherein 15% or more of the microbubbles have a diameter of about 2 microns to less than or about 6 microns.
Clause 60. The method of clause 59, wherein 20% or more of the lipid-encapsulated perfluorocarbon gas microbubbles have a diameter of about 2 microns to less than or about 6 microns.
Clause 61. The method of clause 59 or 60, wherein the lipid formulation comprises DPPA, DPPC and PEG-DPPE, optionally in a mole % ratio of about 10 to about 82 to about 8 including 10:82:8, or wherein the lipid formulation comprises DPPE, DPPC and PEG-DPPE, optionally in a mole % ratio of about 10 to about 82 to about 8 including 10:82:8.
Clause 62. The method of any one of clauses 59-61, wherein PEG-DPPE is PEG5000-DPPE.
Clause 63. The method of any one of clauses 59-61, wherein PEG-DPPE is MPEG5000-DPPE
Clause 64. The method of any one of clauses 59-63, wherein the perfluorocarbon gas is perfluoropropane gas, and the lipid-encapsulated perfluorocarbon gas bubbles are lipid-encapsulated perfluoropropane gas bubbles.
Clause 65. The method of any one of clauses 59-64, wherein the lipid formulation is a non-aqueous lipid solution comprising DPPA, DPPC and PEG-DPPE, glycerol and propylene glycol, and less than or equal to 5% water (v/v), or wherein the lipid formulation is a non-aqueous lipid solution comprising DPPE, DPPC and PEG-DPPE, glycerol and propylene glycol, and less than or equal to 5% water (v/v).
Clause 66. The method of clause 65, wherein the lipid formulation has a lipid concentration of about 3.75 mg/ml.
Clause 67. The method of clause 65 or 66, wherein the lipid formulation is activated at a shaking rate of about 6000 rpm for a shaking time of about 75 seconds to about 90 seconds.
Clause 68. The method of clause 65 or 66, wherein the lipid formulation is activated at a shaking rate of about 6000 rpm for a shaking time of about 75 seconds or about 90 seconds.
Clause 69. The method of clause 65, wherein the lipid formulation has a lipid concentration of about 2.5 mg/ml.
Clause 70. The method of clause 65, wherein the lipid formulation is present in a Wheaton 2 mL glass vial having an internal volume of about 3.8 ml, and wherein the vial contains about 0.35 ml of lipid formulation and about 3.45 ml of gas, or wherein the vial contains about 0.52 ml of lipid formulation and about 3.28 ml of gas.
Clause 71. The method of clause 65, wherein the lipid formulation is present in a Wheaton 2 mL glass vial having an internal volume of about 3.8 ml, wherein about 9% of the internal volume of the vial is occupied by lipid formulation prior to activation, or wherein about 14% of the internal volume of the vial is occupied by lipid formulation prior to activation.
Clause 72. The method of clause 65, wherein prior to activating, a solution of propylene glycol and glycerol, in equal volume proportions, is combined with the lipid formulation.
Clause 73. The method of any one of clauses 69-72, wherein the lipid formulation is activated at a shaking rate of about 6000 rpm for a shaking time of about 60 seconds.
Clause 74. The method of any one of clauses 59-64, wherein the lipid formulation is an aqueous lipid suspension comprising DPPA, DPPC, PEG-DPPE, glycerol and propylene glycol, or wherein the lipid formulation is an aqueous lipid suspension comprising DPPE, DPPC, PEG-DPPE, glycerol and propylene glycol.
Clause 75. The method of clause 74, wherein the lipid formulation has a lipid concentration of about 0.75 mg/ml.
Clause 76. The method of clause 74 or 75, wherein the lipid formulation is activated at a shaking rate of about 6000 rpm for a shaking time of about 30 seconds.
Clause 77. The method of any one of clauses 59-76, wherein the population of lipid-encapsulated perfluorocarbon gas bubbles lacks DPPA.
Clause 78. A population of lipid-encapsulated perfluorocarbon gas bubbles formed according to the method of any one of clauses 59-77.
Clause 79. A method of treating a subject having a condition, comprising
   administering to a subject having a condition an effective amount of a population of lipid-encapsulated perfluorocarbon gas bubbles, wherein 15% or more of microbubbles in the population have a diameter of about 2 microns to less than or about 6 microns, and wherein the lipid-encapsulated perfluorocarbon gas bubbles comprise DPPC and PEG-DPPE, and
   applying ultrasound energy, optionally at a frequency of about 1 to about 10 MHz, to a region of the subject affected by the condition.
Clause 80. In a method of improving therapeutic or diagnostic ultrasound, the improvement comprising
   administering to the subject a population of lipid-encapsulated perfluorocarbon gas bubbles wherein 15% or more of the microbubbles in the population have a diameter in the range of about 2 microns to less than or about 6 microns, before or during exposure to ultrasound, optionally at a frequency of about 1 to about 10 MHz.
Clause 81. The method of clause 79, wherein the condition is cardiac atherosclerosis, myocardial infarct, vascular occlusion, stroke, peripheral artery disease.
Clause 82. The method of clause 79, wherein the lipid-encapsulated perfluorocarbon gas bubbles experience stable cavitation in vivo after application of the ultrasound energy, optionally as detected and/or measured with harmonic frequency emission.
Clause 83. The method of clause 79, wherein the lipid-encapsulated perfluorocarbon gas bubbles experience inertial cavitation in vivo after application of the ultrasound energy, optionally as detected and/or measured with broadband emission.
Clause 84. The method of any one of clauses 79-83, wherein 20% or more of the lipid-encapsulated perfluorocarbon gas microbubbles have a diameter of about 2 microns to less than or about 6 microns.
Clause 85. The method of any one of clauses 79-84, wherein the lipid-encapsulated perfluorocarbon gas bubbles comprise DPPA, DPPC and PEG-DPPE, optionally in a mole % ratio of about 10 to about 82 to about 8 including 10:82:8, or wherein the lipid formulation comprises DPPE, DPPC and PEG-DPPE, optionally in a mole % ratio of about 10 to about 82 to about 8 including 10:82:8.
Clause 86. The method of any one of clauses 79-85, wherein PEG-DPPE is PEG5000-DPPE.
Clause 87. The method of any one of clauses 79-85, wherein PEG-DPPE is MPEG5000-DPPE.
Clause 88. The method of any one of clauses 79-87, wherein the lipid-encapsulated perfluorocarbon gas bubbles are lipid-encapsulated perfluoropropane gas bubbles.
Clause 89. The method of any one of clauses 79-88, wherein the population of lipid-encapsulated perfluorocarbon gas bubbles is formed according to the method of any one of clauses 59-77.
Clause 90. The method of any one of clauses 79-89, wherein the population of lipid-encapsulated perfluorocarbon gas bubbles has an acoustic attenuation, at 2.25 MHz ultrasound frequency, of greater than about 1.0.
Clause 91. The method of any one of clauses 79-89, wherein the population of lipid-encapsulated perfluorocarbon gas microspheres has an acoustic attenuation, at 2.25 MHZ ultrasound frequency, of about 1.0 to about 1.5, or about 1.0 to about 2.0.
Clause 92. The method of any one of clauses 79-90, wherein the population of lipid-encapsulated perfluorocarbon gas bubbles is a composition of any one of clauses 46-58.
Clause 93. A composition comprising
   a population of lipid-encapsulated perfluorocarbon gas bubbles, wherein 4% or more of microbubbles in the population have a diameter of about 6 microns to about 10 microns, and
   wherein the lipid-encapsulated perfluorocarbon gas bubbles comprise DPPC and PEG-DPPE, wherein the microbubbles having a diameter in the range of about 6 microns to about 10 microns are present at a concentration range of about 0.1×10⁸ to about 0.32×10⁸ such bubbles per mL.
Clause 94. The composition of clause 93, wherein 10% or more of the lipid-encapsulated perfluorocarbon gas microbubbles in the population have a diameter of about 6 microns to about microns.
Clause 95. The composition of clause 93 or 94, wherein the lipid-encapsulated perfluorocarbon gas bubbles comprise DPPA, DPPC and PEG-DPPE, optionally in a mole % ratio of about 10 to about 82 to about 8 including 10:82:8, or wherein the lipid-encapsulated perfluorocarbon gas bubbles comprise DPPE, DPPC and PEG-DPPE, optionally in a mole % ratio of about 10 to about 82 to about 8 including 10:82:8.
Clause 96. The composition of any one of clauses 93-95, wherein PEG-DPPE is PEG5000-DPPE.
Clause 97. The composition of any one of clauses 93-96, wherein PEG-DPPE is MPEG5000-DPPE.
Clause 98. The composition of any one of clauses 93-97, wherein lipid-encapsulated perfluorocarbon gas bubbles are lipid-encapsulated perfluoropropane gas bubbles.
Clause 99. The composition of any one of clauses 93-98, wherein the population of lipid-encapsulated perfluorocarbon gas bubbles is present in a non-aqueous lipid solution comprising DPPA, DPPC and PEG-DPPE, glycerol, propylene glycol, and less than or equal to 5% water (v/v), or wherein the population of lipid-encapsulated perfluorocarbon gas bubbles is present in a non-aqueous lipid solution comprising DPPE, DPPC and PEG-DPPE, glycerol, propylene glycol, and less than or equal to 5% water (v/v).
Clause 100. The composition of any one of clauses 93-99, wherein the lipids are present in a combined concentration of about 3.75 mg/ml.
Clause 101. The composition of any one of clauses 93-99, wherein the lipids are present in a combined concentration of about 2.5 mg/ml.
Clause 102. The composition of any one of clauses 93-98, wherein the population of lipid-encapsulated perfluorocarbon gas bubbles is present in an aqueous lipid suspension comprising glycerol and propylene glycol.
Clause 103. The composition of clause 102, wherein the lipids are present in a combined lipid concentration of about 0.75 mg/ml.
Clause 104. The composition of any one of clauses 93-103, wherein the population of lipid-encapsulated perfluorocarbon gas bubbles exhibits an acoustic attenuation of 2.25 MHz ultrasound frequency in the range of greater than about 1, optionally greater than about 1.2, optionally in the range of about 1 to about 1.7.
Clause 105. The composition of any one of clauses 93-104, wherein the population of lipid-encapsulated perfluorocarbon gas bubbles lacks DPPA.
Clause 106. A method for forming lipid-encapsulated perfluorocarbon gas bubbles comprising
   activating a lipid formulation comprising DPPC and PEG-DPPE, glycerol and propylene glycol, in the presence of a perfluorocarbon gas, at a shaking rate and a shaking time sufficient to yield a population of lipid-encapsulated perfluorocarbon gas microbubbles wherein 4% or more of the microbubbles have a diameter of about 6 microns to about 10 microns.
Clause 107. The method of clause 106, wherein 10% or more of the lipid-encapsulated perfluorocarbon gas microbubbles have a diameter of about 6 microns to about 10 microns.
Clause 108. The method of clause 106 or 107, wherein the lipid formulation comprises DPPA, DPPC and PEG-DPPE, optionally in a mole % ratio of about 10 to about 82 to about 8 including 10:82:8, or wherein the lipid formulation comprises DPPE, DPPC and PEG-DPPE, optionally in a mole % ratio of about 10 to about 82 to about 8 including 10:82:8.
Clause 109. The method of any one of clauses 106-108, wherein PEG-DPPE is PEG5000-DPPE.
Clause 110. The method of any one of clauses 106-108, wherein PEG-DPPE is MPEG5000-DPPE.
Clause 111. The method of any one of clauses 106-110, wherein the perfluorocarbon gas is perfluoropropane gas, and the lipid-encapsulated perfluorocarbon gas bubbles are lipid-encapsulated perfluoropropane gas bubbles.
Clause 112. The method of any one of clauses 106-111, wherein the lipid formulation is a non-aqueous lipid solution comprising DPPA, DPPC and PEG-DPPE, glycerol and propylene glycol, and less than or equal to 5% water (v/v), or wherein the lipid formulation is a non-aqueous lipid solution comprising DPPA, DPPC and PEG-DPPE, glycerol and propylene glycol, and less than or equal to 5% water (v/v).
Clause 113. The method of clause 112, wherein the lipid formulation has a lipid concentration of about 3.75 mg/ml.
Clause 114. The method of clause 112, wherein the lipid formulation is present in a Wheaton 2 mL glass vial having an internal volume of about 3.8 ml, and wherein the vial contains about 0.35 ml of lipid formulation and about 3.45 ml of gas, or wherein the vial contains about 0.52 ml of lipid formulation and about 3.28 ml of gas.
Clause 115. The method of clause 112, wherein the lipid formulation is present in a Wheaton 2 mL glass vial having an internal volume of about 3.8 ml, wherein about 9% of the internal volume of the vial is occupied by lipid formulation prior to activation, or wherein about 14% of the internal volume of the vial is occupied by lipid formulation prior to activation.
Clause 116. The method of clause 112, wherein prior to activating, a solution of propylene glycol and glycerol, in equal volume proportions, is combined with the lipid formulation.
Clause 117. The method of clause 112, wherein the lipid formulation has a lipid concentration of about 2.5 mg/ml.
Clause 118. The method of any one of clauses 114-117, wherein the lipid formulation is activated at a shaking rate of about 6000 rpm for a shaking time of about 60 seconds.
Clause 119. The method of any one of clauses 106-111, wherein the lipid formulation is an aqueous lipid suspension comprising DPPA, DPPC, PEG-DPPE, glycerol and propylene glycol, or wherein the lipid formulation is an aqueous lipid suspension comprising DPPE, DPPC, PEG-DPPE, glycerol and propylene glycol.
Clause 120. The method of clause 119, wherein the lipid formulation has a lipid concentration of about 0.75 mg/ml.
Clause 121. The method of any one of clauses 106-120, wherein the population of lipid-encapsulated perfluorocarbon gas bubbles lacks DPPA.
Clause 122. A population of lipid-encapsulated perfluorocarbon gas bubbles formed according to the method of any one of clauses 106-121.
Clause 123. A method of treating a subject having a condition, comprising
   administering to a subject having a condition an effective amount of a population of lipid-encapsulated perfluorocarbon gas bubbles, wherein 4% or more of microbubbles in the population have a diameter of about 6 microns to about 10 microns, and wherein the lipid-encapsulated perfluorocarbon gas bubbles comprise DPPC and PEG-DPPE, and applying ultrasound energy at a frequency of about 1 to about 10 MHz to a region of the subject affected by the condition.
Clause 124. In a method of improving therapeutic or diagnostic ultrasound, the improvement comprising
   administering to the subject a population of lipid-encapsulated perfluorocarbon gas bubbles wherein 4% or more of the microbubbles in the population have a diameter in the range of about 6 microns to about 10 microns, before or during exposure to ultrasound at a frequency of about 1 to about 10 MHz.
Clause 125. The method of clause 124, wherein the condition is cardiac atherosclerosis, myocardial infarct, vascular occlusion, stroke, peripheral artery disease.
Clause 126. The method of clause 124, wherein the lipid-encapsulated perfluorocarbon gas bubbles experience stable cavitation in vivo after application of the ultrasound energy.
Clause 127. The method of clause 124, wherein the lipid-encapsulated perfluorocarbon gas bubbles experience inertial cavitation in vivo after application of the ultrasound energy.
Clause 128. The method of any one of clauses 124-127, wherein 10% or more of the lipid-encapsulated perfluorocarbon gas microbubbles have a diameter of about 6 microns to about 10 microns.
Clause 129. The method of any one of clauses 124-128, wherein the lipid-encapsulated perfluorocarbon gas bubbles comprise DPPA, DPPC and PEG-DPPE, optionally in a mole % ratio of about 10 to about 82 to about 8 including 10:82:8, or wherein the lipid formulation comprises DPPE, DPPC and PEG-DPPE, optionally in a mole % ratio of about 10 to about 82 to about 8 including 10:82:8.
Clause 130. The method of any one of clauses 124-129, wherein PEG-DPPE is PEG5000-DPPE.
Clause 131. The method of any one of clauses 124-129, wherein PEG-DPPE is MPEG5000-DPPE,
Clause 132. The method of any one of clauses 124-131, wherein the lipid-encapsulated perfluorocarbon gas bubbles are lipid-encapsulated perfluoropropane gas bubbles.
Clause 133. The method of any one of clauses 124-132, wherein the population of lipid-encapsulated perfluorocarbon gas bubbles is formed according to the method of any one of clauses 106-121.
Clause 134. The method of any one of clauses 124-133, wherein the population of lipid-encapsulated perfluorocarbon gas bubbles has an acoustic attenuation of 2.25 MHz ultrasound frequency of greater than about 1.0.
Clause 135. The method of any one of clauses 124-133, wherein the population of lipid-encapsulated perfluorocarbon gas microspheres has an acoustic attenuation of 2.25 MHz ultrasound frequency of about 1.0 to about 1.5, or about 1.0 to about 2.0.
Clause 136. A method of ultrasound imaging a subject, comprising
   administering to a subject a population of lipid-encapsulated perfluoropropane gas bubbles of any one of clauses 46-58, 78, 93-105 or 122, or made by the method of any one of clauses 59-77 or 106-121,
   applying ultrasound energy at a frequency in the range of about 1 to about 10 MHZ,
   and obtaining an ultrasound image of the subject or a region of the subject.
Clause 137. The method of clause 136, wherein the ultrasound energy is at a frequency of about 1.25 MHz.
Clause 138. A method for forming lipid-encapsulated perfluoropropane gas bubbles comprising
   activating, in the presence of a gas, a non-aqueous lipid formulation comprising less than or equal to 5% water (v/v), DPPC, PEG-DPPE, glycerol, propylene glycol, and either DPPA or DPPE, at a shaking rate of about 6000 rpm for about 30 to about 90 seconds, optionally for about 50, about 60, about 75, or about 90 seconds, thereby forming a population of lipid-encapsulated perfluoropropane gas microspheres.
Clause 139. The method of clause 138, wherein the lipid formulation and gas are present in a vial, optionally a 2 mL vial, and the lipid formulation occupies about 9% or about 14% of the internal volume of the vial.
Clause 140. A method for forming lipid-encapsulated perfluoropropane gas bubbles comprising
   activating, in the presence of a gas, a non-aqueous lipid formulation comprising less than or equal to 5% water (v/v), DPPC, PEG-DPPE, glycerol, propylene glycol, and either DPPA or DPPE, at a shaking rate of about 4950 rpm for about 125 seconds, thereby forming a population of lipid-encapsulated perfluoropropane gas microspheres.
Clause 141. The method of clause 140, wherein the lipid formulation and gas are present in a vial, optionally a 2 mL vial, and the lipid formulation occupies about 9% or about 14% of the internal volume of the vial.
Clause 142. A method for forming lipid-encapsulated perfluoropropane gas bubbles comprising
   activating an aqueous lipid formulation comprising DPPC and PEG-DPPE, and glycerol and propylene glycol, and either DPPA or DPPE, at a shaking rate of about 6000 rpm for about to about 90 seconds, optionally for about 5, about 10, about 20, about 25, about 30, about 40, about 45, about 50, about 55, about 60, about 70, about 80, or about 90 seconds, thereby forming a population of lipid-encapsulated perfluoropropane gas microspheres.
Clause 143. A method for forming lipid-encapsulated perfluoropropane gas bubbles comprising
   activating an aqueous lipid formulation comprising DPPC and PEG-DPPE, glycerol and propylene glycol, and either DPPA or DPPE, at a shaking rate of about 4530 rpm for about 90 seconds, thereby forming a population of lipid-encapsulated perfluoropropane gas microspheres.
Clause 144. A method for forming lipid-encapsulated perfluoropropane gas bubbles comprising
   activating an aqueous lipid formulation comprising DPPC and PEG-DPPE, glycerol and propylene glycol, and either DPPA or DPPE, at a shaking rate of about 4530 rpm for about 10 to about 20 seconds, thereby forming a population of lipid-encapsulated perfluoropropane gas microspheres.
Clause 145. A method for forming lipid-encapsulated perfluororpropane nanodroplets comprising
   condensing a population of lipid-encapsulated perfluoropropane gas microspheres by applying sufficient pressure to condense the microbubbles into nanodroplets, wherein the population of gas microspheres are condensed at room temperature.
Clause 146. The method of clause 145, wherein applying pressure compriscs applying manual pressure.
Clause 147. The method of clause 145 or 146, wherein applying pressure comprises applying pressure in the range of about 6 to about 12 atm.
Clause 148. The method of any one of clauses 145-147, wherein the population of lipid-encapsulated perfluoropropane gas microspheres is provided in a syringe, optionally a 3 mL syringe, and applying pressure comprises applying manual pressure through a plunger.
Clause 149. The method of any one of clauses 145-148, wherein the population of lipid-encapsulated perfluoropropane gas microspheres is prepared by activating a lipid formulation comprising DPPA, DPPC and PEG5000-DPPE in the presence of perfluoropropane.
Clause 150. The method of any one of clauses 145-148, wherein the population of lipid-encapsulated perfluoropropane gas microspheres is prepared by activating a lipid formulation comprising DPPC, DPPE and PEG5000-DPPE in the presence of perfluoropropane.
Clause 151. The method of clause 149 or 150, wherein the lipid formulation is a non-aqueous lipid formulation and optionally wherein activating a lipid formulation comprises activating at about 4950 rpm for 45 seconds, or about 6000 rpm to about 6200 rpm for about 60 seconds, or about 6600 rpm for about 15 seconds to about 30 seconds.
Clause 152. The method of clause 149 or 150, wherein the lipid formulation is an aqueous lipid formulation and optionally wherein activating a lipid formulation comprises activating at about 4530 rpm for 45 seconds.
Clause 153. A method of diagnosis or therapy in a subject comprising
administering to a subject a population of lipid-encapsulated nanodroplets and exposing the subject to ultrasound energy, optionally at a targeted location, wherein the population of lipid-encapsulated nanodroplets is prepared using the method of any one of clauses 145-152.
Clause 154. The method of clause 153, wherein the nanodroplets comprise a lipid shell made of DPPA, DPPC and PEG5000-DPPE and a liquid center of perfluoropane.
Clause 155. The method of clause 153, wherein the nanodroplets comprise a lipid shell made of DPPC, DPPE and PEG5000-DPPE and a liquid center of perfluoropane,
Clause 156. The method of clause 155, wherein the nanodroplets lack DPPA.
Clause 157. The method of any one of clauses 153-156, wherein the nanodroplets are about 100 to about 200 nanometers in size.
Clause 158. A method of treating a subject having erectile dysfunction comprising
administering to a subject in need thereof an effective amount of lipid-encapsulated gas bubbles or nanodroplets, and
performing ultrasound on the genital area of the subject.
Clause 159. The method of clause 158, wherein the subject having erectile dysfunction has mild-to-moderate erectile dysfunction as measured by an IIEF-5 or an IIEF-15 score.
Clause 160. The method of clause 158 or 159, wherein treatment is evidenced by an increase in an IIEF-5 or an IEF-15 score.
Clause 161. The method of any one of clauses 158-160, wherein the subject is administered lipid-encapsulated gas bubbles that comprise a lipid shell comprising DPPA, DPPC and MPEG5000-DPPE and a perfluorpropane gas center, optionally wherein the lipid-encapsulated gas bubbles are present in an activated DEFINITY formulation or an activated DEFINITY RT formulation.
Clause 162. The method of any one of clauses 158-160, wherein the subject is administered lipid-encapsulated gas bubbles that comprise a lipid shell comprising DPPC, DPPE and MPEG5000-DPPE and a perfluorpropane gas center.
Clause 163. The method of any one of clauses 158-160, wherein the subject is administered lipid-encapsulated nanodroplets that comprise a lipid shell comprising DPPA, DPPC and MPEG5000-DPPE and a center comprising perfluorpropane in liquid form, optionally wherein the lipid-encapsulated nanodroplets are formed by condensation of lipid-encapsulated gas bubbles present in an activated DEFINITY formulation or an activated DEFINITY RT formulation.
Clause 164. The method of any one of clauses 158-160, wherein the subject is administered lipid-encapsulated nanodroplets that comprise a lipid shell comprising DPPC, DPPE and MPEG5000-DPPE and a center comprising perfluorpropane in liquid form.
Clause 165. The method of any one of clauses 158-164, wherein ultrasound comprises high intensity ultrasound, optionally at a frequency in the range of 1 to 2 MHZ, and further optionally at a mechanical index of about 1 to about 1.5 or about 1 to about 1.3.
Clause 166. The method of any one of clauses 158-165, wherein the method is performed repeatedly at regularly spaced intervals, optionally wherein the method is performed once or twice a week for one or more weeks.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “cithor,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited. In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A composition comprising
a population of lipid-encapsulated perfluorocarbon gas bubbles, wherein 75% or more of the lipid-encapsulated perfluorocarbon gas bubbles are nanobubbles having a diameter in the range of about 100 nanometers to less than or about 1 micron, and wherein the lipid-encapsulated perfluorocarbon gas bubbles comprise DPPC and PEG-DPPE, optionally wherein the nanobubbles are present in a concentration range of about 7.0×10⁹ to about 2.5×10¹⁰ nanobubbles per mL, or about 1×10¹⁰ to about 2.5×10¹⁰ nanobubbles per mL.

2. A method for forming the lipid-encapsulated perfluorocarbon gas bubbles of claim 1 comprising
activating a lipid formulation comprising DPPC and PEG-DPPE, glycerol and propylene glycol, in the presence of a perfluorocarbon gas, at a shaking rate and shaking time sufficient to yield a population of lipid-encapsulated perfluorocarbon gas bubbles wherein 75% or more of the gas bubbles are nanobubbles having a diameter in the range of about 100 nanometers to about 1 micron.

3. (canceled)

4. A method of treating a subject having a condition, comprising
administering to a subject having a condition an effective amount of the population of lipid-encapsulated perfluorocarbon gas bubbles of claim 1, wherein 75% or more of the bubbles in the population are nanobubbles having a diameter in the range of about 100 nanometers to about 1 micron, and wherein the lipid-encapsulated perfluorocarbon gas bubbles comprise DPPC and PEG-DPPE, and
applying ultrasound energy to a region of the subject affected by the condition.

5. In a method of improving tissue perfusion in a subject comprising exposing the subject to therapeutic ultrasound, the improvement comprising
administering to the subject the population of lipid-encapsulated perfluorocarbon gas bubbles of claim 1, wherein 75% or more of the bubbles in the population are nanobubbles having a diameter in the range of about 100 nanometers to about 1 micron before and/or during exposure to the therapeutic ultrasound.

6. A composition comprising
a population of lipid-encapsulated perfluorocarbon gas bubbles, wherein 15% or more of microbubbles in the population have a diameter of about 2 microns to less than or about 6 microns, and
wherein the lipid-encapsulated perfluorocarbon gas bubbles comprise DPPC and PEG-DPPE, optionally wherein the microbubbles having a diameter of about 2 microns to less than or about 6 microns are present in a concentration range of about 0.24×10⁹ to about 2.44×10⁹ bubbles per mL.

7. A method for forming the lipid-encapsulated perfluorocarbon gas bubbles of claim 6 comprising
activating a lipid formulation comprising DPPC and PEG-DPPE, glycerol and propylene glycol, in the presence of a perfluorocarbon gas, at a shaking rate and a shaking time sufficient to yield a population of lipid-encapsulated perfluorocarbon gas microbubbles wherein 15% or more of the microbubbles have a diameter of about 2 microns to less than or about 6 microns.

8. (canceled)

9. A method of treating a subject having a condition, comprising
administering to a subject having a condition an effective amount of the population of lipid-encapsulated perfluorocarbon gas bubbles of claim 6, wherein 15% or more of microbubbles in the population have a diameter of about 2 microns to less than or about 6 microns, and wherein the lipid-encapsulated perfluorocarbon gas bubbles comprise DPPC and PEG-DPPE, and
applying ultrasound energy, optionally at a frequency of about 1 to about 10 MHz, to a region of the subject affected by the condition.

10. In a method of improving therapeutic or diagnostic ultrasound, the improvement comprising
administering to the subject the population of lipid-encapsulated perfluorocarbon gas bubbles of claim 6, wherein 15% or more of the microbubbles in the population have a diameter in the range of about 2 microns to less than or about 6 microns, before or during exposure to ultrasound, optionally at a frequency of about 1 to about 10 MHz.

11. A composition comprising
a population of lipid-encapsulated perfluorocarbon gas bubbles, wherein 4% or more of microbubbles in the population have a diameter of about 6 microns to about 10 microns, and
wherein the lipid-encapsulated perfluorocarbon gas bubbles comprise DPPC and PEG-DPPE, wherein the microbubbles having a diameter in the range of about 6 microns to about 10 microns are present at a concentration range of about 0.1×10⁸ to about 0.32×10⁸ such bubbles per mL.

12. A method for forming the lipid-encapsulated perfluorocarbon gas bubbles of claim 11 comprising
activating a lipid formulation comprising DPPC and PEG-DPPE, glycerol and propylene glycol, in the presence of a perfluorocarbon gas, at a shaking rate and a shaking time sufficient to yield a population of lipid-encapsulated perfluorocarbon gas microbubbles wherein 4% or more of the microbubbles have a diameter of about 6 microns to about 10 microns.

13. (canceled)

14. A method of treating a subject having a condition, comprising
administering to a subject having a condition an effective amount of the population of lipid-encapsulated perfluorocarbon gas bubbles of claim 11, wherein 4% or more of microbubbles in the population have a diameter of about 6 microns to about 10 microns, and wherein the lipid-encapsulated perfluorocarbon gas bubbles comprise DPPC and PEG-DPPE, and
applying ultrasound energy at a frequency of about 1 to about 10 MHz to a region of the subject affected by the condition.

15. In a method of improving therapeutic or diagnostic ultrasound, the improvement comprising
administering to the subject the population of lipid-encapsulated perfluorocarbon gas bubbles of claim 11, wherein 4% or more of the microbubbles in the population have a diameter in the range of about 6 microns to about 10 microns, before or during exposure to ultrasound at a frequency of about 1 to about 10 MHz.

16. A method of ultrasound imaging a subject, comprising
administering to a subject a population of lipid-encapsulated perfluoropropane gas bubbles of claim 6,
applying ultrasound energy at a frequency in the range of about 1 to about 10 MHZ,
and obtaining an ultrasound image of the subject or a region of the subject.

17. A method for forming lipid-encapsulated perfluoropropane gas bubbles comprising
activating, in the presence of a gas, a non-aqueous lipid formulation comprising less than or equal to 5% water (v/v), DPPC, PEG-DPPE, glycerol, propylene glycol, and either DPPA or DPPE
(a) at a shaking rate of about 6000 rpm for about 30 to about 90 seconds, optionally for about 50, about 60, about 75, or about 90 seconds; or
(b) at a shaking rate of about 4950 rpm for about 125 seconds,
thereby forming a population of lipid-encapsulated perfluoropropane gas bubbles.

18. (canceled)

19. A method for forming lipid-encapsulated perfluoropropane gas bubbles comprising
activating an aqueous lipid formulation comprising DPPC and PEG-DPPE, and glycerol and propylene glycol, and either DPPA or DPPE (a)
(a) at a shaking rate of about 6000 rpm for about 5 to about 90 seconds, optionally for about 5, about 10, about 20, about 25, about 30, about 40, about 45, about 50, about 55, about 60, about 70, about 80, or about 90 seconds;
(b) at a shaking rate of about 4530 rpm for about 90 seconds; or
(c) at a shaking rate of about 4530 rpm for about 10 to about 20 seconds,
thereby forming a population of lipid-encapsulated perfluoropropane gas bubbles.

20-21. (canceled)

22. A method for forming lipid-encapsulated perfluororpropane nanodroplets comprising
condensing a population of lipid-encapsulated perfluoropropane gas microspheres by applying sufficient pressure to condense the microbubbles into nanodroplets, wherein the population of gas microspheres are condensed at room temperature.

23. A method of diagnosis or therapy in a subject comprising
administering to a subject a population of lipid-encapsulated nanodroplets and exposing the subject to ultrasound energy, optionally at a targeted location, wherein the population of lipid-encapsulated nanodroplets is prepared using the method of claim 22.

24. A method of treating a subject having erectile dysfunction comprising
administering to a subject in need thereof an effective amount of lipid-encapsulated gas bubbles or nanodroplets, and
performing ultrasound on the genital area of the subject.
